(19) United States
(12) Patent Application Publication
(10) Pub. No.: US 2011/0190324 A1
(43) Pub. Date: Aug. 4, 2011

(54) METHODS OF TREATING ATHEROSCLEROSIS

(76) Inventor: Edward Leung, Cary, NC (US)

(21) Appl. No.: 12/997,963

(22) PCT Filed: Jul. 15, 2009
(86) PCT No.: PCT/US09/50626

§ 371 Date: Jan. 24, 2011

Related U.S. Application Data
(60) Provisional application No. 61/081,235, filed on Jul. 16, 2008.

(51) Int. Cl. A61K 31/519; A61P 9/10

(52) U.S. Cl. 514/267

(57) ABSTRACT

The present invention relates to adenosine A3 receptor antagonists and their use for the prevention and treatment of atherosclerosis by administering to a mammal, in need thereof, a therapeutically effective amount of an adenosine A3 receptor antagonist, or a pharmaceutically acceptable salt thereof, alone or in combination with other anti-atherosclerotic agents.

### FIELD OF THE INVENTION

The present invention relates to adenosine A₃ receptor antagonists and their use for the prevention and treatment of atherosclerosis by administering to a mammal, in need thereof, a therapeutically effective amount of an adenosine A₃ receptor antagonist, or a pharmaceutically acceptable salt thereof, alone or in combination with other anti-atherosclerotic agents.

### BACKGROUND OF THE INVENTION

Cardiovascular disease is a leading cause of morbidity and mortality, particularly in the United States and in Western European countries. Atherosclerosis, the most prevalent of cardiovascular diseases, is the principle cause of heart attack, stroke and vascular circulation problems. Atherosclerosis is a complex disease which involves many cell types, biochemical events and molecular factors. Several causative factors are implicated in the development of cardiovascular disease including hereditary predisposition to the disease, gender, lifestyle factors such as smoking and diet, age, hypertension, and hyperlipidemia, including hypercholesterolemia. Several of these factors, particularly hyperlipidemia and hypercholesterolemia (high blood cholesterol concentrations) provide a significant risk factor associated with atherosclerosis.

Cholesterol is present in the blood as free and esterified cholesterol within lipoprotein particles, commonly known as chylomicrons, very low density lipoproteins (VLDLs), low density lipoproteins (LDLs), and high density lipoproteins (HDLs). Concentration of total cholesterol in the blood is influenced by (1) absorption of cholesterol from the digestive tract, (2) synthesis of cholesterol from dietary constituents such as carbohydrates, proteins, fats and ethanol, and (3) removal of cholesterol from blood by tissues, especially the liver, and subsequent conversion of the cholesterol to bile acids, steroid hormones, and biliary cholesterol. The formation of macrophage foam cells, by cholesterol accumulation, is the key event in the development of atherosclerosis.

Maintenance of blood cholesterol concentrations is influenced by both genetic and environmental factors. Genetic factors include concentration of rate-limiting enzymes in cholesterol biosynthesis, concentration of receptors for low density lipoproteins in the liver, concentration of rate-limiting enzymes for conversion of cholesterols bile acids, rates of synthesis and secretion of lipoproteins and gender of person. Environmental factors influencing the hemostasis of blood cholesterol concentration in humans include dietary composition, incidence of smoking, physical activity, and use of a variety of pharmaceutical agents. Dietary variables include amount and type of fat (saturated and polyunsaturated fatty acids), amount of cholesterol, amount and type of fiber, and perhaps amounts of vitamins such as vitamin C and D and minerals such as calcium.

Clinical studies have firmly established that the elevated plasma concentrations of LDL are associated with accelerated atherogenesis, i.e., formation of atherosclerotic lesions.

On the other hand, it is well understood that hypertension is a leading cause of cardiovascular diseases such as stroke, heart attack, heart failure and irregular heart beat. Hypertension is a condition where the pressure of blood within the blood vessels is higher than normal as it circulates through the body. When the systolic pressure exceeds 150 mmHg or the diastolic pressure exceeds 90 mmHg for a sustained period of time, damage is done to the body. For example, excessive systolic pressure can rupture blood vessels anywhere, and when it occurs within the brain, a stroke results. Hypertension may also cause thickening and narrowing of the blood vessels which ultimately could lead to atherosclerosis.

However, reduction of high blood pressure has an effect on coronary mortality and morbidity lower than expected. One of the possible explanations is the different anti-atherogenic capacity of anti-hypertensive drugs. Reduction of high blood pressure has, by itself, an anti-atherogenic effect, but, for some anti-hypertensive drugs, there is experimental and clinical evidence of anti-atherogenic properties beyond blood pressure lowering, e.g., for calcium antagonists, experimental data have been published reporting reduction of aortic lipidic deposition and decrease of arterial proliferation.

Adenosine exerts a number of physiological functions through activation of four cell membrane receptors classified as A₁, A_{2A}, A_{2B} and A₃. The most recently discovered subtype, the A₃ subtype, has been the subject of intensive pharmacological characterization. Although all adenosine subclasses belong to the G protein-coupled receptors they are associated with different second messenger systems. The A₃ subtype is believed to have a characteristic second messenger profile, in that it has been shown to mediate adenylyl cyclase inhibition and phospholipase C activation.

The adenosine A₃ receptor is believed to play a role in modulation of cerebral ischemia, inflammation, hypertension, ischemic heart pre-conditioning and asthma. This has made the A₃ receptor as an attractive new therapeutic target. For example, selective antagonists for the A₃ receptor have been proposed for use as anti-inflammatory and anti-ischemic agents in the brain. Furthermore, A₃ antagonists have been under development as anti-agiogenetic (cancer), anti-asthmatic, anti-depressant, anti-arrhythmic, renal protective and anti-parkinson's agents, and cognitive enhancing drugs.

### SUMMARY OF THE INVENTION

Surprisingly, it has now been discovered that adenosine A₃ receptor antagonists may be employed for the prevention and treatment of atherosclerosis, independent of the anti-hypertensive effect of adenosine A₃ antagonists, by preventing and slowing the progression of atherosclerotic plaque build-up. Thus, adenosine A₃ receptor antagonists may also be employed for the prevention of stroke and heart attack. More surprisingly, it has been demonstrated that adenosine A₃ receptor antagonists may be employed for the regression of atherosclerotic plaque.

Accordingly, the present invention provides a method for the prevention and treatment of atherosclerosis, and the subsequent prevention stroke and heart attack, which method comprises administering to a mammal a therapeutically effective amount of an adenosine A₃ receptor antagonist, or a pharmaceutically acceptable salt thereof, alone or in combination with other therapeutic agents.

Adenosine A₃ receptor antagonists to be employed in the methods of the present invention include, but are not limited to, compounds of the formula

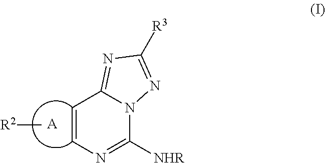

wherein

A, R, R² and R³ have the meaning as described herein in the Detailed Description of the Invention, or a pharmaceutically acceptable salt thereof.

Other objects, features, advantages and aspects of the present invention will become apparent to those skilled in the art from the following description and appended claims. It should be understood, however, that the following description, appended claims, and specific examples, while indicating preferred embodiments of the invention, are given by way of illustration only. Various changes and modifications within the spirit and scope of the disclosed invention will become readily apparent to those skilled in the art from reading the following.

### BRIEF DESCRIPTION OF THE DRAWINGS

FIGS. 1A, 1B, 1C and 1D show mRNA and protein expression of adenosine A₁, A_{2A}, A_{2B} and A₃ receptors, respectively, in PMA-treated U937 cells, human macrophages (HM) and foam cells (FC) under normoxic (N) and hypoxic (H) conditions. The expression level of adenosine A_{2B} receptors is normalized to the expression level of the endogenous reference (β-actin) in each sample.

FIGS. 2A, 2B, 2C and 2D show a Western blot analysis of the expression of adenosine A₁, A_{2A}, A_{2B} and A₃ receptors, respectively, in PMA-treated U937 cells, human macrophages (HM) and foam cells (FC) under normoxic (N) and hypoxic (H) conditions. Cellular extracts were prepared and subjected to immunoblot assay using anti-A₁, A_{2A}, A_{2B} and A₃ antibodies. Tubulin shows equal loading of protein.

FIGS. 3A, 3B, 3C and 3D show Bmax (fmol/mg of protein) of human A₁, A_{2A}, A_{2B} and A₃ adenosine receptors, respectively, as evaluated through binding studies. Values are the means and vertical lines represent S.E. of the mean of four separate experiments, each performed in triplicate.

FIGS. 4A, 4B, 4C, 4D, 4E, 4F and 4G show the effect of 100 μM adenosine on HIF-1α in PMA-treated U937 cells, human macrophages (HM) and foam cells (FC) under normoxia (N) (FIGS. 4A, 4C and 4E, respectively) and hypoxia (H) (FIGS. 4B, 4D, 4F and 4G). U937 cells were treated with 50 and 100 μg of oxLDL (FIGS. 4E, 4G and 4F). HIF-1β shows equal loading of protein. Densitometric quantification of HIF-1α western blots is the mean±S.E. values (N=3); *P<0.05 compared with the control.

FIG. 5 shows the effect of adenosine (100 μM) on HIF-1α accumulation and antagonism by 100 nM MRE-3008F20, SCH 58261, DPCPX and MRE-2029F20. Densitometric quantification of HIF-1α western blots is the mean±S.E. values (N=3).

FIG. 6 shows the accumulation of HIF-1α in the absence (column 1) and in the presence of adenosine receptor agonists: 10 and 100 nM CHA (columns 2, 3); 500 and 1000 nM CGS 21680 (columns 4, 5); 10 and 100 nM 1-deoxy-1-[6-{4-[(phenylcarbamoyl)-methoxy]phenylamino}-9H-purin-9-yl]-N-ethyl-β-D-ribofuranuronamide (columns 6,7); 10 and 100 nM CI-IB-MECA (columns 8, 9). Densitometric quantification of HIF-1α western blots is the mean±S.E. values (N=3); P<0.05 compared with the control.

FIGS. 7A, 7B, 7C, 7D, 7E, 7F, 7G, 7H and 7I show adenosine receptor silencing by siRNA transfection in foam cells (FC). Relative adenosine receptor mRNA quantification, related to β-actin mRNA, by real-time RT-PCR. Foam cells were transfected with siRNA of A₁, A_{2A}, A_{2B} and A₃ adenosine receptors (FIGS. 7A, 7B, 7C and 7D, respectively) and cultured for 24, 48 and 72 h. Plots are mean±S.E. values (N=3); *P<0.01 compared with the control (time=0). Western blot analysis using anti-A₁, A_{2A}, A_{2B} and A₃ receptor polyclonal antibodies (FIGS. 7E, 7F, 7G and 7H, respectively) of protein extracts from foam cells treated with siRNAs of each adenosine receptor subtype and cultured for 24, 48 and 72 h. Tubulin shows equal loading of protein. FIG. 7I shows the effect of adenosine on HIF-1α modulation in the absence (column 2) and in the presence of siRNA of A₁, A_{2A}, A_{2B} or A₃ adenosine receptors (columns 3, 4, 5, 6, respectively), and in the presence of siRNA of A₁, A_{2A}, A_{2B} and A₃ adenosine receptors together (siAdoRs) (column 7). Densitometric quantification of western blots is the mean±S.E. values (N=3); *P<0.05 compared with the control (column 1) (72 h scramble-transfected cells).

FIG. 8 shows the effect of adenosine on VEGF secretion. Foam cells were treated with 100 μM adenosine in the absence and in the presence of 100 nM DPCPX, SCH 58261, MRE-3008F20 or MRE-2029F20. Bargraphs are the means and vertical lines represent S.E. of the mean of four separate experiments, each performed in triplicate; *P<0.05 compared with the control or 72 h scramble-transfected cells (−siRNA).

FIG. 9 shows the effect of adenosine on IL-8 secretion. Foam cells were treated with 100 μM adenosine in the absence and in the presence of 100 nM DPCPX, SCH 58261, MRE-3008F20 or MRE-2029F20. Bargraphs are the means and vertical lines represent S.E. of the mean of four separate experiments performed in triplicate; P<0.05 compared with the control or 72 h scramble-transfected cells (−siRNA).

FIGS. 10A, 10B, 10C and 10D show the inhibition of foam cell formation from PMA-treated U937 cells in the presence of oxLDL and adenosine, by addition of the adenosine A₃ receptor antagonist MRE-3008F20. Cells are stained for lipids with Oil red O in parallel cultures by incubation in the absence (FIG. 10A) and the presence of oxLDL (50 μg/mL), but in the absence of adenosine (FIG. 10B), or in the presence of oxLDL (50 μg/mL) and adenosine (100 μM, FIG. 10C), at 37° C. for 24 h followed by paraformaldehyde fixation. FIG. 10D shows the effect of the A₃ receptor antagonist MRE-3008F20 (100 nM) on oxLDL and adenosine induced foam cells formation.

FIGS. 11A, 11B and 11C show the inhibition of foam cell formation from PMA-treated U937 cells in the presence of oxLDL and adenosine, by addition of the adenosine A₃ receptor antagonist VUF 5574. Cells are stained for lipids with Oil red O in parallel cultures by incubation in the presence of oxLDL (50 μg/mL) but in the absence of adenosine (FIG. 11A), or in the presence of oxLDL (50 μg/mL) and adenosine (100 μM, FIG. 11B), at 37° C. for 24 h followed by paraformaldehyde fixation. FIG. 11C shows the effect of the A₃ receptor antagonist VUF 5574 (10 nM) on oxLDL and adenosine induced foam cells formation.

FIGS. 12A, 12B, 12C and 12D show the inhibition of foam cell formation from PMA-treated U937 cells in the presence of oxLDL and adenosine, by addition of the adenosine A_{2B} receptor antagonist MRE-2029F20. Cells are stained for lipids with Oil red O in parallel cultures by incubation in the absence (FIG. 12A) and the presence of oxLDL (50 μg/mL), but in the absence of adenosine (FIG. 12B), or in the presence of oxLDL (50 μg/mL) and adenosine (100 μM, FIG. 12C), at 37° C. for 24 h followed by paraformaldehyde fixation.

FIG. 12D shows the effect of the A_{2B} receptor antagonist MRE-2029F20 (100 nM) on oxLDL and adenosine induced foam cells formation.

### DETAILED DESCRIPTION OF THE INVENTION

As noted herein above, macrophage foam cell formation is an important process in the development of atherosclerotic lesions and plaque. Atherosclerosis is initiated by dysfunction of endothelial cells at lesion-prone sites in the walls of arteries and results in monocyte infiltration into the arterial intima. These cells then differentiate into macrophages which ingest large amounts of oxidized LDL (oxLDL), slowly turning into large cholesterol-loaded “foam cells”. Under a microscope, the lesions now appear as fatty streaks in the arterial wall. As the atherosclerotic lesions progress, the arterial wall thickness increases and oxygen diffusion into the intima is markedly reduced. These hypoxic regions contain a large number of foam cells revealing that these cells experience hypoxia during the development of atherosclerotic lesions and plaque. Indeed, it has been suggested that an imbalance between the demand and supply of oxygen in the arterial wall is a key factor for the development of atherosclerotic lesions (Bjornheden et al., Arterioscler, Thromb. Vasc., 19: 870-876, 1999).

Hypoxia-inducible factor-1 (HIF-1), the most important factor involved in the cellular response to hypoxia, is an heterodimeric transcription factor composed of an inducibly-expressed HIF-1α subunit and a constitutively-expressed HIF-1β subunit (Semenza et al., Trends Mol. Med., 7: 345-350, 2001). It has been reported that oxLDL induce hypoxia-inducible factor-1 (HIF-1) accumulation in human Mono-Mac-6 macrophages suggesting that HIF-1 may play a role in atherosclerosis. It is well established that HIF-1 plays a major role in vascular endothelial growth factor (VEGF) expression and angiogenesis with the notion that VEGF mediates important alterations associated with atherogenesis and angiogenic activity of macrophages. Recent finding suggest that neovascularization within atherosclerotic plaques is a sign of advanced atherosclerosis/restenosis (Shatrov et al., Blood, 101: 4847-4849, 2003). Furthermore it has been reported that under atherogenic conditions high expression of HIF-1 in macrophages promotes foam cell formation and atherosclerosis (Jiang et al., Eur. J. Pharmacol., 562: 183-190, 2007).

Foam cells isolated from human atherosclerotic tissue display elevated levels of another potent angiogenic agent, interleukin-8 (CXCL8, IL-8). Recently, CXCL8 has been shown to be up-regulated by foam cells found in hypoxic zones in rabbit and human atherosclerotic plaques. It has been suggested that hypoxia-induced secretion of CXCL8 from foam cells may lead to the recruitment of smooth muscle, vascular endothelial and T-cells into the atherosclerotic plaques and, thus, to plaque progression. Neovascularization is a key characteristic of tissue pathology in all stages of atherosclerosis and cancer.

The purine nucleoside adenosine has been consensually identified as a major local regulator of tissue function especially when energy supply fails to meet cellular energy demand, thus, earning in the 1980s the reputation of retaliatory metabolite (Newby A. C., Trends Biol. Sci., 9: 42-44, 1984). Adenosine levels appear to reach very high levels during hypoxia, ischemia, inflammation and injury. Under these conditions, adenosine is released into the extracellular space and signals through the activation of extracellular G-protein coupled adenosine receptors, namely, the adenosine A₁, A_{2A}, A_{2B}, and A₃ receptor subtypes. It has been demonstrated that adenosine, through activation of A₃ receptors, induces HIF-1α accumulation under hypoxic conditions in certain cancer cell lines, and subsequently increases VEGF levels, suggesting a potential role of adenosine in cancer angiogenesis (Merighi et al., Biochem. Pharmacol., 72: 19-31, 2006; Merighi et al., Mol. Pharmacol., 72: 395-406, 2007). Furthermore, it has been recently reported that in murine macrophages activation of adenosine A_{2A} receptor subtypes induces accumulation of HIF-1α and VEGF, whereas increased levels of VEGF in monocytes was found to be related to A₁ receptor activation (De Ponti et al., J. Leukoc. Biol., 82: 392-402, 2007; Ramanathan et al., Molecular Biology of the Cell, 18, 14-23, 2007).

Surprisingly, it has now been discovered that the adenosine A₃ receptor stimulates hypoxia induced transformation of macrophages into foam cells. Furthermore, it has been discovered that adenosine A₃ receptor antagonists may be employed to block the formation of foam cells. Thus, adenosine A₃ receptor antagonists may be employed for the prevention and treatment of atherosclerosis by preventing and slowing the progression of atherosclerotic plaque build-up, and subsequently preventing stroke and heart attack. More surprisingly, it has been demonstrated that adenosine A₃ receptor antagonists may be employed for the regression of atherosclerotic plaque.

Accordingly, the present invention provides a method for the inhibition of foam cell formation and, thus, a method for the prevention and treatment of atherosclerosis, and the subsequent prevention of stroke and heart attack, which method comprises administering to a mammal, in need thereof, a therapeutically effective amount of an adenosine A₃ receptor antagonist, or a pharmaceutically acceptable salt thereof.

Furthermore, the present invention provides a combination therapy for the prevention and treatment of atherosclerosis, and the subsequent prevention of stroke and heart attack, comprising an adenosine A₃ receptor antagonist in combination with at least one other therapeutic agent selected from the group consisting of (1) an angiotensin converting enzyme (ACE) inhibitor; (2) an angiotensin II receptor blocker; (3) a renin inhibitor; (4) a diuretic; (5) a calcium channel blocker (CCB); (6) a beta-blocker; (7) a platelet aggregation inhibitor; (8) a cholesterol absorption modulator; (9) a HMG-Co-A reductase inhibitor; (10) a high density lipoprotein (HDL) increasing compound; (11) acyl-CoA:cholesterol O-acyltransferase (ACAT) inhibitor; and (12) an adenosine A_{2B} receptor antagonist; or in each case, a pharmaceutically acceptable salt thereof.

In other words, the present invention provides a method for the prevention and treatment of atherosclerosis, and the subsequent prevention of stroke and heart attack, which method comprises administering to a mammal, in need thereof, a therapeutically effective amount of a combination of an adenosine A₃ receptor antagonist, or a pharmaceutically acceptable salt thereof, and at least one other therapeutic agent selected from the group consisting of:

(1) an ACE inhibitor;

(2) an angiotensin II receptor blocker;

(3) a renin inhibitor;

(4) a diuretic;

(5) a calcium channel blocker;

(6) a beta-blocker;

(7) a platelet aggregation inhibitor;

(8) a cholesterol absorption modulator;

(9) a HMG-Co-A reductase inhibitor;

(10) a high density lipoprotein (HDL) increasing compound;

(11) an ACAT inhibitor; and

(12) an adenosine A_{2B} receptor antagonist;

or in each case, a pharmaceutically acceptable salt thereof.

Listed below are some of the definitions of various terms used herein to describe certain aspects of the present invention. However, the definitions used herein are those generally known in the art and apply to the terms as they are used throughout the specification unless they are otherwise limited in specific instances.

The term “prevention” refers to prophylactic administration to healthy patients to prevent the development of the conditions mentioned herein above.

The term “treatment” is understood the management and care of a patient for the purpose of combating the disease, condition or disorder, e.g., the progression of atherosclerotic plaque build-up.

The term “therapeutically effective amount” refers to an amount of a drug or a therapeutic agent that will elicit the desired biological or medical response of a tissue, system or an animal (including man) that is being sought by a researcher or clinician.

The term “mammal or patient” are used interchangeably herein and include, but are not limited to, humans, dogs, cats, horses, pigs, cows, monkeys, rabbits, mice and laboratory animals. The preferred mammals are humans.

The term “pharmaceutically acceptable salt” refers to a non-toxic salt commonly used in the pharmaceutical industry which may be prepared according to methods well-known in the art. Pharmaceutically acceptable salts of the compounds employed in the present invention refer to salts formed with acids, namely acid addition salts, such as of mineral acids, organic carboxylic acids and organic sulfonic acids, e.g., hydrochloric acid, maleic acid and methanesulfonic acid, respectively. Similarly, pharmaceutically acceptable salts of the compounds employed in the invention refer to salts formed with bases, namely cationic salts, such as alkali and alkaline earth metal salts, e.g., sodium, lithium, potassium, calcium and magnesium, as well as ammonium salts, e.g., ammonium, trimethylammonium, diethylammonium and tris(hydroxymethyl)-methyl-ammonium salts and salts with amino acids provided an acidic group constitutes part of the structure.

The term “combination” of an adenosine A₃ receptor antagonist, and another therapeutic agent(s) referred to herein above, or in each case, a pharmaceutically acceptable salt thereof, means that the components can be administered together as a pharmaceutical composition or as part of the same, unitary dosage form. A combination also includes administering an adenosine A₃ receptor antagonist, or a pharmaceutically acceptable salt thereof, and another therapeutic agent(s) referred to herein above, or in each case, a pharmaceutically acceptable salt thereof, each separately but as part of the same therapeutic regimen. The components, if administered separately, need not necessarily be administered at essentially the same time, although they can if so desired. Thus, a combination also refers, e.g., administering an adenosine A₃ receptor antagonist, or a pharmaceutically acceptable salt thereof, and another therapeutic agent(s), or in each case, a pharmaceutically acceptable salt thereof, as separate dosages or dosage forms, but at the same time. A combination also includes separate administration at different times and in any order.

As used herein, the term “alkyl” refers to a monovalent straight or branched saturated hydrocarbon group preferably having from 1 to 20 carbon atoms, more preferably 1 to 10 carbon atoms (“lower alkyl”) and most preferably 1 to 6 carbon atoms. This term is exemplified by groups such as methyl, ethyl, n-propyl, i-propyl, n-butyl, i-butyl, n-hexyl, and the like. The terms “alkylene” and “lower alkylene” refer to divalent radicals of the corresponding alkane. Further, as used herein, other moieties having names derived from alkanes, such as alkoxy, alkanoyl, alkenyl etc. when modified by “lower,” have carbon chains of ten or less carbon atoms. In those cases where the minimum number of carbons is greater than one, e.g., alkenyl (minimum of two carbons), it is to be understood that “lower” means at least the minimum number of carbons.

As used herein, the term “substituted alkyl” refers to an alkyl group, preferably of from 1 to 10 carbon atoms (“substituted lower alkyl”), having from 1 to 5 substituents, and preferably 1 to 3 substituents, selected from the group consisting of alkoxy, cycloalkyl, cycloalkenyl, acyl, acylamino, acyloxy, amino, aminoacyl, aminoacyloxy, cyano, halogen, hydroxy, keto, thioketo, carboxy, carboxyalkyl, thiol, alkylthio, aryl, aryloxy, heteroaryl, heteroaryloxy, heterocyclyl, alkoxyamino, nitro, —SO-alkyl, —SO-aryl, —SO-heteroaryl, —SO₂-alkyl, —SO₂-aryl, —SO₂-heteroaryl, and mono- and dialkylamino, mono- and diarylamino, mono and diheteroarylamino, mono and diheterocyclyl amino, and unsymmetric disubstituted amino groups. As used herein, other moieties having the prefix “substituted” are intended to include one or more of the substituents listed above.

As used herein, the term “cycloalkyl” refers to cyclic alkyl groups of from 3 to 12 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantyl, and the like.

As used herein, “aralkyl” refers to an alkyl group with an aryl substituent. Binding is through the alkyl group. Examples of aralkyl groups include benzyl and phenethyl.

As used herein, the term “alkenyl” refers to an unsaturated, straight or branched hydrocarbon group preferably having from 2 to 10 carbon atoms and more preferably 2 to 6 carbon atoms and having at least one, and preferably from 1 or 2, double bonds. Preferred alkenyl groups include ethenyl (—CH═CH₂), n-propenyl (—CH₂—CH═CH₂), i-propenyl (—C(CH₃)═CH₂), and the like.

As used herein, the term “alkynyl” refers to an unsaturated, straight or branched hydrocarbon group preferably having from 2 to 10 carbon atoms and more preferably 2 to 6 carbon atoms and having at least 1 and preferably from 1 or 2 triple bonds.

As used herein, the term “alkoxy” refers to the group “alkyl-O—”, where alkyl is as defined above. Preferred alkoxy groups include, by way of example, methoxy, ethoxy, n-propoxy, i-propoxy, n-butoxy, t-butoxy, s-butoxy, n-pentyloxy, n-hexyloxy, 1,2-dimethylbutoxy, and the like.

As used herein, the term “alkylthio” refers to the group “alkyl-S—”, where alkyl is as defined above.

As used herein, the term “acyl” refers to the groups alkyl-C(O)— (alkanoyl), substituted alkyl-C(O)—, cycloalkyl-C(O)—, substituted cycloalkyl-C(O)—, aryl-C(O)—, substituted aryl-C(O)—, heteroaryl-C(O)— and heterocyclyl-C(O)— wherein alkyl, substituted alkyl, cycloalkyl, substituted cycloalkyl, aryl, heteroaryl and heterocyclic are as defined herein.

As used herein, the term “aminoacyl” refers to the group —C(O)NR′R″ where R′ and R″ are independently hydrogen, alkyl, substituted alkyl, aryl, substituted aryl, heteroaryl, or heterocyclyl wherein alkyl, substituted alkyl, aryl, heteroaryl and heterocyclyl are as defined herein.

As used herein, the term “acylamino” refers to the group R′C(O)—NR″— wherein R′ and R″ are independently hydrogen, alkyl, substituted alkyl, cycloalkyl, substituted cycloalkyl, aryl, substituted aryl, heteroaryl, or heterocyclyl wherein alkyl, substituted alkyl, cycloalkyl, substituted cycloalkyl, aryl, heteroaryl and heterocyclic are as defined herein.

As used herein, the term “acyloxy” refers to the group R′C(O)—O— where each R′ is alkyl, substituted alkyl, cycloalkyl, substituted cycloalkyl, aryl, substituted aryl, heteroaryl, or heterocyclyl wherein alkyl, substituted alkyl, cycloalkyl, aryl, heteroaryl and heterocyclyl are as defined herein.

As used herein, the term “aryl” refers to an unsaturated aromatic carbocyclic group of from 6 to 14 carbon atoms having a single ring (e.g., phenyl) or multiple condensed (fused) rings (e.g., naphthyl or anthryl). Preferred aryls include phenyl, naphthyl and the like. Unless otherwise constrained by the definition for the aryl substituent, such aryl groups can optionally be substituted with from 1 to 5 substituents, and preferably 1 to 3 substituents, selected from the group consisting of hydroxy, acyl, alkyl, alkoxy, alkenyl, alkynyl, amino, di(lower alkyl)amino, aminoacyl, acyloxy, acylamino, aralkyl, aryl, aryloxy, azido, carboxy, cyano, halo, nitro, heteroaryl, heteroaryloxy, heterocyclyl, heterocyclooxy, alkylthio, —SO-alkyl, —SO-substituted alkyl, —SO-aryl, —SO-heteroaryl, —SO₂-alkyl, —SO₂-substituted alkyl, —SO₂-aryl, and

—SO₂-heteroaryl. Preferred substituents include C₁ to C₄ alkyl, C₁ to C₄ alkoxy, halogen, cyano, nitro, C₁ to C₄ haloalkyl, e.g., trihalomethyl, C₁ to C₄ haloalkoxy, e.g., dihalomethyl, di(lower alkyl)amino, carboxy, and acylamino.

As used herein, the terms “halo” or “halogen” refer to fluoro, chloro, bromo and iodo and preferably is either fluoro or chloro.

As used herein, the term “heteroaryl” refers to an aromatic heterocycle having from 1 to 15 carbon atoms and 1 to 4 heteroatoms selected from the group consisting of oxygen, nitrogen and sulfur within at least one ring (if there is more than one ring).

Unless otherwise constrained by the definition for the heteroaryl substituent, such heteroaryl groups can be optionally substituted with from 1 to 5 substituents, and preferably 1 to 3 substituents, selected from the group consisting of hydroxy, acyl, alkyl, alkoxy, alkenyl, alkynyl, substituted alkyl, substituted alkoxy, substituted alkenyl, substituted alkynyl, amino, di(lower alkyl)amino, aminoacyl, acyloxy, acylamino, alkaryl, aryl, aryloxy, azido, carboxy, cyano, halo, nitro, heteroaryl, heteroaryloxy, heterocyclyl, heterocyclooxy, alkylthio, substituted alkylthio, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, and —SO₂-heteroaryl. Preferred substituents include C₁ to C₄ alkyl, C₁ to C₄ alkoxy, halogen, cyano, nitro, C₁ to C₄ haloalkyl, e.g., trihalomethyl, C₁ to C₄ haloalkoxy, e.g., dihalomethyl, di(lower alkyl)amino, carboxy, and acylamino. Such heteroaryl groups can have a single ring (e.g., pyridyl or furyl) or multiple condensed rings (e.g., indolizinyl or benzothienyl).

“Heterocyclo” or “heterocyclyl” refers to a monovalent saturated or unsaturated heterocyclic group having a single ring or multiple condensed rings, from 1 to 15 carbon atoms and from 1 to 4 hetero atoms selected from the group consisting of nitrogen, sulfur and oxygen within at least one ring (if there is more than one ring).

Unless otherwise constrained by the definition for the heterocyclic group, such heterocyclyl groups can be optionally substituted with 1 to 5 substituents, and preferably 1 to 3 substituents, selected from the group consisting of alkyl, substituted alkyl, alkoxy, substituted alkoxy, aryl, aryloxy, halogen, cyano, nitro, C₁ to C₄ haloalkyl, e.g., trihalomethyl, C₁ to C₄ haloalkoxy, e.g., dihalomethyl, heteroaryl, thiol, alkylthio, amino, di(lower alkyl)amino, carboxy, acylamino, and the like. Such heterocyclic groups can have a single ring or multiple condensed rings.

As used herein, the term “heterocyclooxy” refers to a heterocyclic group bonded through an oxygen bridge.

As to any of the above groups that contain one or more substituents, it is understood, of course, that such groups do not contain any substitution or substitution patterns which are sterically impractical and/or synthetically non-feasible.

Suitable adenosine A₃ receptor antagonists to which the present invention applies include MRS 1191, MRS 1220, MRS 1334, MRS 1523, MRS 3777 hemioxalate, VUF 5574, PSB 10 hydrochloride, PSB 11 hydrochloride and reversine (commercially available from Sigma-Aldrich and/or Tocris Bioscience). Other suitable antagonists include those disclosed in U.S. Pat. No. 6,358,964; U.S. Pat. No. 6,620,825; U.S. Pat. No. 6,673,802; U.S. Pat. No. 6,686,366; U.S. Pat. No. 6,921,825; U.S. Pat. No. 7,064,204; U.S. Pat. No. 7,371,737; and U.S. 20060178385; the entire contents of which are incorporated herein by reference. Additional adenosine receptor antagonists may be found in Jacobson et al., Neuropharmacology, 36: 1157-1165, 1997; Yao et al., Biochem. Biophys. Res. Commun., 232: 317-322, 1997; Kim et al., J. Med. Chem., 39(21): 4142-4148, 1996; van Rhee et al., Drug Devel. Res., 37: 131, 1996; van Rhee et al., J. Med. Chem., 39: 2980-2989, 1996; Siquidi et al., Nucleosides, Nucleotides 15: 693-718, 1996; van Rhee et al., J. Med. Chem., 39: 398-406, 1996; Jacobson et al., Drugs of the Future, 20: 689-699, 1995; Jacobson et al., J. Med. Chem., 38: 1720-1735, 1995; Karton et al., J. Med. Chem., 39: 2293-2301, 1996; Kohno et al., Blood, 88: 3569-3574, 1996; Jiang et al., J. Med. Chem., 39: 4667-4675, 1996; Yao et al., Biochem. Biophys. Res. Commun. 232: 317-322, 1997; and Jiang et al., J. Med. Chem. 40: 2596-2608, 1996.

Optionally, the adenosine A₃ antagonists to be employed in the methods of the present invention may also exhibit antagonistic activity on the other adenosine receptor subtypes, in particular, on the adenosine A_{2B} receptor subtype.

In one aspect, the present invention relates to a method for the inhibition of foam cell formation and, thus, a method for the prevention and treatment of atherosclerosis, and the subsequent prevention of stroke and heart attack, by administering to a mammal, in need thereof, a therapeutically effective amount of an adenosine A₃ receptor antagonist disclosed in U.S. Pat. No. 6,921,825.

More specifically, the present invention provides a method for the inhibition of foam cell formation and, thus, a method for the prevention and treatment of atherosclerosis, and the subsequent prevention of stroke and heart attack, by employing an adenosine A₃ receptor antagonist of the formula

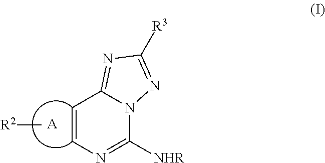

wherein

A is imidazole, pyrazole, or triazole;

R is —C(X)R¹, —C(X)—N(R¹)₂, —C(X)OR¹, —C(X)SR¹, —SO_{b}R¹, —SO_{b}OR¹, —SO_{b}SR¹, or —SO_{b}—N(R¹)₂;

R¹ is hydrogen, alkyl, substituted alkyl, cycloalkyl, substituted cycloalkyl, alkenyl, substituted alkenyl, alkynyl, substituted alkynyl, aryl, substituted aryl, heteroaryl, substituted heteroaryl, heterocyclyl, substituted heterocyclyl, wherein each R¹ can be the same or different; or, if linked to a nitrogen atom, then taken together with the nitrogen atom, —N(R¹)₂ forms an azetidine ring or a 5- or 6-membered heterocyclic ring optionally containing one or more additional heteroatoms selected from the group consisting of N, O, and S;

R² is hydrogen, alkyl, alkenyl, alkynyl, substituted alkyl, substituted alkenyl, substituted alkynyl, aralkyl, substituted aralkyl, aryl, substituted aryl, heteroaryl, or substituted heteroaryl;

R³ is furan, pyrrole, thiophene, benzofuran, benzypyrrole, benzothiophene, optionally substituted with 1 to 3 substituents selected from the group consisting of hydroxy, acyl, alkyl, alkoxy, alkenyl, alkynyl, substituted alkyl, substituted alkoxy, substituted alkenyl, substituted alkynyl, amino, aminoacyl, acyloxy, acylamino, aralkyl, aryl, substituted aryl, aryloxy, azido, carboxy, cyano, halo, nitro, heteroaryl, heteroaryloxy, heterocyclyl, heterocyclooxy, alkylthio, substituted alkylthio, —SO-alkyl, —SO-substituted alkyl, —SO-aryl, —SO-heteroaryl, —SO₂-alkyl,

—SO₂-substituted alkyl, —SO₂-aryl, —SO₂-heteroaryl, and trihalomethyl;

X is O, S, or NR'; and

b is 1 or 2;

or a pharmaceutically acceptable salt thereof.

Preferably, R¹ is hydrogen; C₁ to C₈ alkyl; C₂ to C₇ alkenyl; C₂ to C₇ alkynyl; C₃ to C₇ cycloalkyl; C₁ to C₅ alkyl substituted with 1 to 3 substituents selected from halogen, hydroxy, C₁ to C₄ alkoxy, and C₃ to C₇ cycloalkyl; C₆ to C₁₀ aryl optionally substituted with 1 to 3 substituents selected from C₁ to C₄ alkoxy, C₁ to C₄ alkyl, halogen, cyano, nitro, amino, di(lower alkyl)amino, C₁ to C₄ haloalkyl, C₁ to C₄ haloalkoxy, carboxy, and acylamino; C₇ to C₁₀ aralkyl in which the aryl moiety can be substituted with 1 to 3 of the substituents indicated above for the aryl group; a group of formula —(CH₂)ₘ-Het, in which Het is a 5- to 6-membered aromatic or non-aromatic heterocyclic ring containing one or more heteroatoms selected from the group consisting of N, O, and S, and m is zero, or an integer from 1 to 5; and wherein each R¹ can be the same or different.

More preferably, R¹ is hydrogen, 5- to 6-membered heteroaryl optionally substituted with 1 to 3 substituents selected from the group consisting of C₁ to C₄ alkyl, C₁ to C₄ alkoxy, halogen, cyano, nitro, amino, di(lower alkyl)amino, C₁ to C₄ haloalkyl, C₁ to C₄ haloalkoxy, carboxy, and acylamino; or C₆ to C₁₀ aryl or C₇ to C₁₀ aralkyl wherein, in each case, the aryl group may be optionally substituted as described herein above for aryl; and wherein each R¹ can be the same or different.

Particularly preferred compounds are those in which R¹ is hydrogen, 5- to 6-membered heteroaryl, or a phenyl group, in each case, optionally substituted with 1 to 3 substituents selected from the group consisting of Br, Cl, F, methoxy, nitro, cyano, methyl, trifluoromethyl, difluoromethoxy, and di(lower alkyl)amino; and wherein each R¹ can be the same or different.

Preferred C₁ to C₈ alkyl groups are methyl, ethyl, propyl, butyl and isopentyl. Examples of preferred C₃ to C₇ cycloalkyl groups include cyclopropyl, cyclopentyl, and cyclohexyl. Examples of preferred C₁ to C₅ alkyl groups substituted with C₃ to C₇ cycloalkyl groups include cyclohexylmethyl, cyclopentylmethyl, and 2-cyclopentylethyl. Examples of preferred substituted C₁ to C₅ alkyl groups also include 2-hydroxyethyl, 2-methoxyethyl, trifluoromethyl, 2-fluoroethyl, 2-chloroethyl, 3-aminopropyl, 2-(4-methyl-1-piperazine)ethyl, 2-(4-morpholinyl)ethyl, 2-aminocarbonylethyl, 2-dimethylaminoethyl, and 3-dimethylaminopropyl.

Aryl is preferably phenyl, optionally substituted with 1 to 3 substituents selected from Br, Cl, F, methoxy, nitro, cyano, methyl, trifluoromethyl, difluoromethoxy and di(lower alkyl)amino groups.

Examples of preferred 5- to 6-membered heterocyclic groups containing N, O and/or S include piperazinyl, morpholinyl, thiazolyl, pyrazolyl, pyridyl, furyl, thienyl, pyrrolyl, triazolyl, and tetrazolyl.

Examples of preferred C₇ to C₁₀ aralkyl groups include benzyl or phenethyl in each of which the phenyl group may be optionally substituted by 1 to 3 substituents selected from Br, Cl, F, methoxy, nitro, cyano, methyl, trifluoromethyl, and difluoromethoxy.

Preferably, R² is C₁ to C₈ alkyl optionally substituted with 1 to 3 substituents selected from halogen, hydroxy, C₁ to C₄ alkoxy, and C₃ to C₇ cycloalkyl.

Preferably, R³ is furan, pyrrole, thiophene, benzofuran, indole, benzothiophene, optionally substituted with 1 to 3 substituents selected from the group consisting of alkyl, alkoxy, halo, cyano, nitro, trihalomethyl, and alkylthio.

Preferably, X is O, R² is C₂ to C₃ alkyl optionally substituted with 1 to 3 substituents selected from halogen, hydroxy, C₁ to C₄ alkoxy, and C₃ to C₇ cycloalkyl; and R³ is furyl.

The possible meanings of A may be represented by the following structural formulae:

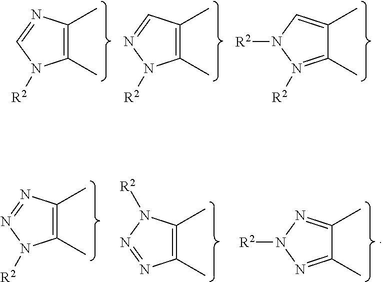

In a specific embodiment of the present invention, the method of the present invention is conducted by administering to a mammal, in need thereof, a therapeutically effective amount of a compound of formula (I), wherein R² is selected from the group consisting of hydrogen, alkyl, alkenyl and aryl, or a pharmaceutically acceptable salt thereof.

In another specific embodiment of the present invention, the method of the present invention is conducted by administering to a mammal, in need thereof, a therapeutically effective amount of a compound of formula (I), wherein A represents an imidazole ring, or a pharmaceutically acceptable salt thereof.

Yet in another specific embodiment of the present invention, the method of the present invention is conducted by administering to a mammal, in need thereof, a therapeutically effective amount of a compound of formula (I), wherein A represents a pyrazole ring. More specifically, A represents a pyrazole ring of the formula

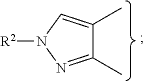

or a pharmaceutically acceptable salt thereof.

Yet in another specific embodiment of the present invention, the method of the present invention is conducted by administering to a mammal, in need thereof, a therapeutically effective amount of a compound of formula (I), wherein A represents a triazole ring, or a pharmaceutically acceptable salt thereof.

Yet in another specific embodiment of the present invention, the method of the present invention is conducted by administering to a mammal, in need thereof, a therapeutically effective amount of a compound of formula (I), wherein R represents —C(X)—N(R¹)₂ in which

R¹ is hydrogen, alkyl, substituted alkyl, alkenyl, substituted alkenyl, alkynyl, substituted alkynyl, aryl, substituted aryl, heteroaryl, substituted heteroaryl, heterocyclyl, substituted heterocyclyl, wherein each R¹ can be the same or different; or, if linked to a nitrogen atom, then taken together with the nitrogen atom, —N(R¹)₂ forms an azetidine ring or a 5- or 6-membered heterocyclic ring optionally containing one or more additional heteroatoms selected from the group consisting of N, O, and S;

X is O;

or a pharmaceutically acceptable salt thereof.

Yet in another specific embodiment of the present invention, the method of the present invention is conducted by administering to a mammal, in need thereof, a therapeutically effective amount of a compound of formula (I), wherein

R represents —C(O)—N(R¹)₂ in which each R¹ is different from each other, one being hydrogen;

A represents a pyrazole ring of the formula

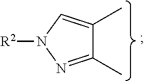

or a pharmaceutically acceptable salt thereof.

Yet in another specific embodiment of the present invention, the method of the present invention is conducted by administering to a mammal, in need thereof, a therapeutically effective amount of a compound of formula (I) having the formula

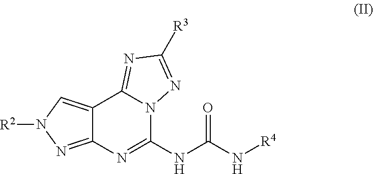

wherein

R² is hydrogen, alkyl, substituted alkyl, alkenyl, aralkyl, substituted aralkyl, heteroaryl, substituted heteroaryl or aryl;

R³ is furan;

R⁴ is aryl, substituted aryl, heteroaryl, substituted heteroaryl, heterocycle or substituted heterocycle;

or a pharmaceutically acceptable salt thereof.

Non-limiting examples of compounds of formulae (I) and (II) include those listed herein below and those depicted in Table 1:
5-{[(3-Chlorophenyl)amino]carbonyl}amino-8-methyl-2-(2-furyl)-pyrazolo[4,3-e]-1,2,4-triazolo[1,5-c]pyrimidine;
5-{[(4-Methoxyphenyl)amino]carbonyl}amino-8-methyl-2-(2-furyl)-pyrazolo[4,3-e]-1,2,4-triazolo[1,5-c]pyrimidine;
5-{[(3-Chlorophenyl)amino]carbonyl}amino-8-ethyl-2-(2-furyl)-pyrazolo[4,3-e]-1,2,4-triazolo[1,5-c]pyrimidine;
5-{[(4-Methoxyphenyl)amino]carbonyl}amino-8-ethyl-2-(2-furyl)-pyrazolo[4,3-e]-1,2,4-triazolo[1,5-c]pyrimidine;
5-{[(3-Chlorophenyl)amino]carbonyl}amino-8-propyl-2-(2-furyl)-pyrazolo[4,3-e]-1,2,4-triazolo[1,5-c]pyrimidine;
5-{[(4-Methoxyphenyl)amino]carbonyl}amino-8-propyl-2-(2-furyl)-pyrazolo[4,3-e]-1,2,4-triazolo[1,5-c]pyrimidine;
5-{[(3-Chlorophenyl)amino]carbonyl}amino-8-butyl-2-(2-furyl)-pyrazolo[4,3-e]-1,2,4-triazolo[1,5-c]pyrimidine;
5-{[(4-Methoxyphenyl)amino]carbonyl}amino-8-butyl-2-(2-furyl)-pyrazolo[4,3-e]-1,2,4-triazolo[1,5-c]pyrimidine;
5-{[(3-Chlorophenyl)amino]carbonyl}amino-8-isopentyl-2-(2-furyl)-pyrazolo[4,3-e]-1,2,4-triazolo[1,5-c]pyrimidine;
5-{[(4-Methoxyphenyl)amino]carbonyl}amino-8-isopentyl-2-(2-furyl)-pyrazolo[4,3-e]-1,2,4-triazolo[1,5-c]pyrimidine;
5-{[(3-Chlorophenyl)amino]carbonyl}amino-8-(2-isopentenyl)-2-(2-furyl)pyrazolo[4,3-e]-1,2,4-triazolo[1,5-c]pyrimidine;
5-{[(4-Methoxyphenyl)amino]carbonyl}amino-8-(2-isopentenyl)-2-(2-furyl)-pyrazolo[4,3-e]-1,2,4-triazolo[1,5-c]pyrimidine;
5-{[(3-Chlorophenyl)amino]carbonyl}amino-8-(2-phenylethyl)-2 (2-furyl)-pyrazolo[4,3-e]-1,2,4-triazolo[1,5-c]pyrimidine;
5-{[(4-Methoxyphenyl)amino]carbonyl}amino-8-(2-phenylethyl)-2-(2-furyl)-pyrazolo[4,3-e]-1,2,4-triazolo[1,5-c]pyrimidine;
5-{[(3-Chlorophenyl)amino]carbonyl}amino-8-(3-phenylpropyl)-2-(2-furyl)-pyrazolo[4,3-e]-1,2,4-triazolo[1,5-c]pyrimidine;
5-{[(4-Methoxyphenyl)amino]carbonyl}amino-8-(3-phenylpropyl)-2-(2-furyl)-pyrazolo[4,3-e]-1,2,4-triazolo[1,5-c]pyrimidine;
5-[(Benzyl)carbonyl]amino-8-isopentyl-2-(2-furyl)-pyrazolo[4,3-e]-1,2,4-triazolo[1,5-c]pyrimidine;
5-[(Benzyl)carbonyl]amino-8-(3-phenylpropyl)-2-(2-furyl)-pyrazolo[4,3-e]-1,2,4-triazolo[1,5-c]pyrimidine;
N-[4-(Diethylamino)phenyl]-N′-[2-(2-furyl)-8-methyl-8H-pyrazolo[4,3-e]-1,2,4-triazolo[1,5-c]pyrimidin-5-yl]urea;
N-[8-Methyl-2-(2-furyl)-8H-pyrazolo[4,3-e]-1,2,4-triazolo[1,5-c]pyrimidin-5-yl]-N′-[4-(dimethylamino)phenyl]urea;
N-[2-(2-Furyl)-8-methyl-8H-pyrazolo[4,3-e]-1,2,4-triazolo[1,5-c]pyrimidin-5-yl]-N′-[4-(morpholin-4-ylsulfonyl)phenyl]urea;
N-[2-(2-Furyl)-8-methyl-8H-pyrazolo[4,3-e]-1,2,4-triazolo[1,5-c]pyrimidin-5-yl]-N′-{4-[(4-methylpiperazin-1-yl)sulfonyl]phenyl}urea; and
N-[2-(2-Furyl)-8-methyl-8H-pyrazolo[4,3-e]-1,2,4-triazolo[1,5-c]pyrimidin-5-yl]-N′-pyridin-4-ylurea;
   or a pharmaceutically acceptable salt thereof.

TABLE 1

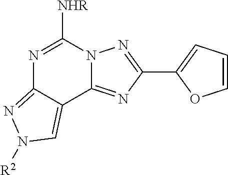

| R² | R |
|---|---|
| H | 4-MeO—Ph—NHCO— |
| H | 3-Cl—Ph—NHCO— |
| t-C₄H₉ | 4-MeO—Ph—NHCO— |
| t-C₄H₉ | 3-Cl—Ph—NHCO— |
| CH₃ | Ph—NHCO— |
| CH₃ | 4-SO₃H—Ph—NHCO— |
| CH₃ | 3,4-Cl₂—Ph—NHCO— |
| CH₃ | 3,4-(OCH₂—O)—Ph—NHCO— |
| CH₃ | 4-(NO₂)—Ph—NHCO— |
| CH₃ | 4-(CH₃)—Ph—NHCO— |
| CH₃ | Ph—(CH₂)—CO— |
| C₂H₅ | Ph—NHCO— |
| C₂H₅ | 4-SO₃H—Ph—NHCO— |
| C₂H₅ | 3,4-Cl₂—Ph—NHCO— |
| C₂H₅ | 3,4-(OCH₂—O)—Ph—NHCO— |
| C₂H₅ | 4-(NO₂)—Ph—NHCO— |
| C₂H₅ | 4-(CH₃)—Ph—NHCO— |
| C₂H₅ | Ph—(CH₂)CO— |
| n-C₃H₇ | Ph—NHCO— |
| n-C₃H₇ | 4-SO₃H—Ph—NHCO— |
| n-C₃H₇ | 3,4-Cl₂—Ph—NHCO— |
| n-C₃H₇ | 3,4-(OCH₂—O)—Ph—NHCO— |
| n-C₃H₇ | 4-(NO₂)—Ph—NHCO— |
| n-C₃H₇ | 4-(CH₃)—Ph—NHCO— |
| n-C₃H₇ | Ph—(CH₂)CO— |
| n-C₄H₉ | Ph—NHCO— |
| n-C₄H₉ | 4-SO₃H—Ph—NHCO— |
| n-C₄H₉ | 3,4-Cl₂—Ph—NHCO— |
| n-C₄H₉ | 3,4-(OCH₂—O)—Ph—NHCO— |
| n-C₄H₉ | 4-(NO₂)—Ph—NHCO— |
| n-C₄H₉ | 4-(CH₃)—Ph—NHCO— |
| 2-(α-napthyl)ethyl | Ph—(CH₂)—CO— |
| 2-(α-napthyl)ethyl | 4-MeO—Ph—NHCO— |
| 2-(α-napthyl)ethyl | 3-Cl—Ph—NHCO— |
| 2-(2,4,5-tribromophenyl)ethyl | 4-MeO—Ph—NHCO— |
| 2-(2,4,5-tribromophenyl)ethyl | 3-Cl—Ph—NHCO— |
| 2-propen-1-yl | 4-MeO—Ph—NHCO— |

Preferred are compounds of formula (II), especially those selected from the group consisting of:

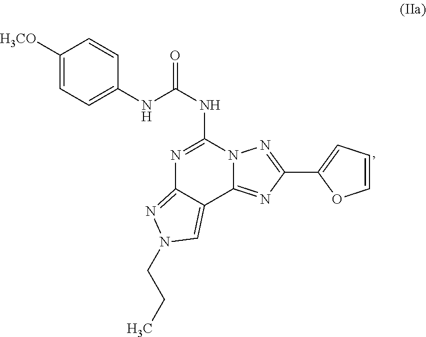

5-[[(4-methoxyphenyl)amino]carbonyl]amino-8-propyl-2-(2-furyl)-pyrazolo[4,3-e]-1,2,4-triazolo[1,5-c]pyrimidine, also known as MRE-3008F20, or a pharmaceutically acceptable salt thereof;

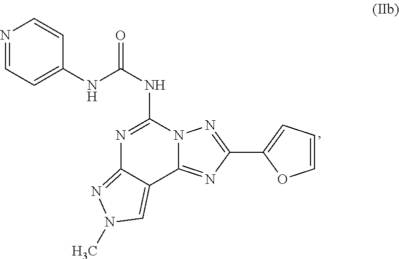

N-[2-(2-Furyl)-8-methyl-8H-pyrazolo[4,3-e]-1,2,4-triazolo[1,5-c]pyrimidin-5-yl]-N′-pyridin-4-ylurea, or a pharmaceutically acceptable salt thereof, in particular the hydrochloride salt thereof;

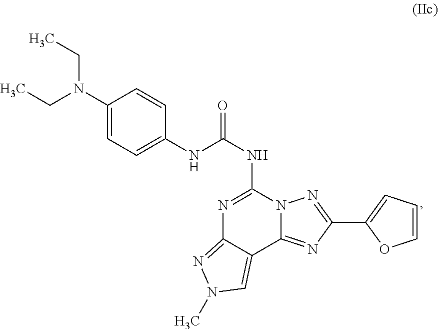

N-1-(4-diethylamino-phenyl)-N′-S-[8-methyl-2-(2-furyl)-pyrazolo[4,3-e]-1,2,4-triazolo[1,5-c]pyrimidine]urea, or a pharmaceutically acceptable salt thereof; and

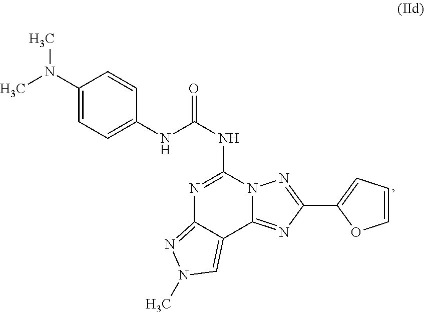

N-1-(4-dimethylamino-phenyl)-N′-5-[8-methyl-2-(2-furyl)-pyrazolo[4,3-e]-1,2,4-triazolo[1,5-c]pyrimidine]urea, or a pharmaceutically acceptable salt thereof.

In another aspect, the present invention relates to a method for the inhibition of foam cell formation and, thus, a method for the prevention and treatment of atherosclerosis, and the subsequent prevention of stoke and heart attack, by administering to a mammal, in need thereof, a therapeutically effective amount of an adenosine A₃ receptor antagonist disclosed in U.S. Pat. No. 6,358,964.

More specifically, the present invention provides a method for the inhibition of foam cell formation and, thus, a method for the prevention and treatment of atherosclerosis, and the subsequent prevention of stroke and heart attack, by employing an adenosine A₃ receptor antagonist of the formula

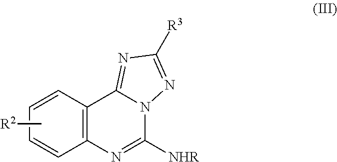

wherein

R is —C(X)R¹, —C(X)—N(R¹)₂, —C(X)OR¹, —C(X)SR¹, —SO_{b}R¹, —SO_{b}OR¹, —SO_{b}SR¹, or —SO_{b}—N(R¹)₂;

R¹ is hydrogen, alkyl, substituted alkyl, alkenyl, substituted alkenyl, alkynyl, substituted alkynyl, aryl, heteroaryl, substituted heteroaryl, or heterocyclyl, wherein each R¹ may be the same or different; or, if linked to a nitrogen atom, then taken together with the nitrogen atom,

—N(R¹)₂ forms an azetidine ring or a 5- to 6-membered heterocyclic ring optionally containing one or more heteroatoms selected from N, O, and S;

R² is hydrogen, halogen, alkyl, alkenyl, alkynyl, substituted alkyl, substituted alkenyl, substituted alkynyl, aralkyl, substituted aralkyl, aryl, substituted aryl, heteroaryl or substituted heteroaryl;

R³ is furan, pyrrole, thiophene, benzofuran, benzypyrrole, benzothiophene, optionally substituted with 1 to 3 substituents selected from the group consisting of hydroxy, acyl, alkyl, alkoxy, alkenyl, alkynyl, substituted alkyl, substituted alkoxy, substituted alkenyl, substituted alkynyl, amino, aminoacyl, acyloxy, acylamino, alkaryl, aryl, substituted aryl, aryloxy, azido, carboxy, cyano, halo, nitro, heteroaryl, heteroaryloxy, heterocyclyl, heterocyclooxy, thioalkyl, substituted thioalkyl, —SO-alkyl, —SO-substituted alkyl, —SO-aryl, —SO-heteroaryl, —SO₂-alkyl,

—SO₂-substituted alkyl, —SO₂-aryl, —SO₂-heteroaryl, and trihalomethyl;

X is O, S, or NR¹;

b is 1 or 2;

or a pharmaceutically acceptable salt thereof.

Preferably, for compounds of formula (III), R¹ is hydrogen; C₁ to C₈ alkyl; C₂ to C₇ alkenyl; C₂ to C₇ alkynyl; C₃ to C₇ cycloalkyl; C₁ to C₅ alkyl substituted with 1 to 3 substituents selected from halogen, hydroxy, C₁ to C₄ alkoxy, and C₃ to C₇ cycloalkyl; C₆ to C₁₀ aryl optionally substituted with 1 to 3 substituents selected from C₁ to C₄ alkoxy, C₁ to C₄ alkyl, halogen, cyano, nitro, amino, di(lower alkyl)amino, C₁ to C₄ haloalkyl, C₁ to C₄ haloalkoxy, carboxy, and acylamino; C₇ to C₁₀ aralkyl in which the aryl moiety can be substituted with 1 to 3 of the substituents indicated above for the aryl group; a group of formula —(CH₂)ₘ-Het, in which Het is a 5- to 6-membered aromatic or non-aromatic heterocyclic ring containing one or more heteroatoms selected from the group consisting of N, O, and S, and m is zero, or an integer from 1 to 5; and wherein each R¹ can be the same or different.

More preferably, for compounds of formula (III), R¹ is hydrogen, 5- to 6-membered heteroaryl optionally substituted with 1 to 3 substituents selected from the group consisting of C₁ to C₄ alkyl, C₁ to C₄ alkoxy, halogen, cyano, nitro, amino, di(lower alkyl)amino, C₁ to C₄ haloalkyl, C₁ to C₄ haloalkoxy, carboxy, and acylamino; or C₆ to C₁₀ aryl or C₇ to C₁₀ aralkyl wherein, in each case, the aryl group may be optionally substituted as described herein above for aryl; and wherein each R¹ can be the same or different.

Particularly preferred compounds of formula (III) are those in which R¹ is 5- to 6-membered heteroaryl, or a phenyl group optionally substituted with 1 to 3 substituents selected from the group consisting of Br, Cl, F, methoxy, nitro, cyano, methyl, trifluoromethyl, difluoromethoxy or di(lower alkyl)amino groups; and wherein each R¹ can be the same or different.

For compounds of formula (III), preferred C₁ to C₈ alkyl groups are methyl, ethyl, propyl, butyl and isopentyl. Examples of preferred C₃ to C₇ cycloalkyl groups include cyclopropyl, cyclopentyl, and cyclohexyl. Examples of preferred C₁ to C₅ alkyl groups substituted with C₃ to C₇ cycloalkyl groups include cyclohexylmethyl, cyclopentylmethyl, and 2-cyclopentylethyl. Examples of preferred substituted C₁ to C₅ alkyl groups also include 2-hydroxyethyl, 2-methoxyethyl, trifluoromethyl, 2-fluoroethyl, 2-chloroethyl, 3-aminopropyl, 2-(4-methyl-1-piperazine)ethyl, 2-(4-morpholinyl)ethyl, 2-aminocarbonylethyl, 2-dimethylaminoethyl, and 3-dimethylaminopropyl.

For compounds of formula (III), aryl is preferably phenyl, optionally substituted with one or more substituents selected from Br, Cl, F, methoxy, nitro, cyano, methyl, trifluoromethyl, difluoromethoxy and di(lower alkyl)amino groups.

For compounds of formula (III), examples of preferred 5 to 6-membered ring heterocyclic groups containing N, O and/or S include piperazinyl, morpholinyl, thiazolyl, pyrazolyl, pyridyl, furyl, thienyl, pyrrolyl, triazolyl, and tetrazolyl.

For compounds of formula (III), examples of preferred C₇ to C₁₀ aralkyl groups comprise benzyl or phenethyl optionally substituted by one or more substituents selected from Br, Cl, F, methoxy, nitro, cyano, methyl, trifluoromethyl, and difluoromethoxy.

Preferably, for compounds of formula (III), R² is halogen, preferably chloro, C₂ to C₃ alkyl or substituted C₂ to C₃ alkyl.

Preferably, for compounds of formula (III), R³ isfuran, pyrrole, thiophene, benzofuran, indole, benzothiophene, optionally substituted with 1 to 3 substituents selected from the group consisting of alkyl, alkoxy, halo, cyano, nitro, trihalomethyl, and thioalkyl.

Preferably, for compounds of formula (III), X is O, R² is chloro, and R³ is furan.

In a specific embodiment of the present invention, the method of the present invention is conducted by administering to a mammal, in need thereof, a therapeutically effective amount of a compound of formula (III), wherein R represents —C(X)—N(R¹)₂ in which X is O.

Non-limiting examples of compounds of formula (III) include those listed herein below:
5-{[4-Methoxyphenyl)amino]carbonyl}amino-9-chloro-2-(2-furyl)-1,2,4-triazolo[1,5-c]quinazoline; and
5-{[3-Chlorophenyl)amino]carbonyl}amino-9-chloro-2-(2-furyl)-1,2,4-triazolo[1,5-c]quinazoline;
   or a pharmaceutically acceptable salt thereof.

Yet in another aspect, the present invention relates to a method for the inhibition of foam cell formation and, thus, a method for the prevention and treatment of atherosclerosis, and the subsequent prevention of stroke and heart attack, by administering to a mammal, in need thereof, a therapeutically effective amount of an adenosine A₃ receptor antagonist disclosed in U.S. Patent Application Publication No. 20060178385.

More specifically, the present invention provides a method for the inhibition of foam cell formation and, thus, a method for the prevention and treatment of atherosclerosis, and the subsequent prevention of stroke and heart attack, by employing an adenosine A₃ receptor antagonist of the formula

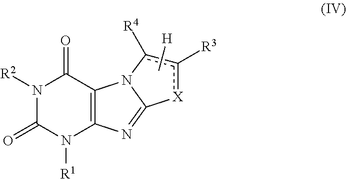

wherein

X is CH or N;

R¹ and R² are each independently hydrogen, alkyl, substituted alkyl, aralkyl, substituted aralkyl, alkenyl, substituted alkenyl, alkynyl, substituted alkynyl, aryl, or substituted aryl;

R³ is aryl, substituted aryl, alkyl, substituted alkyl, aralkyl, substituted aralkyl;

R⁴ is hydrogen, alkyl, substituted alkyl, aralkyl, substituted aralkyl, aryl, or substituted aryl; and

one of the dashed lines represents a double bond and the other represents a single bond;

or a pharmaceutically acceptable salt thereof.

Preferably, for compounds of formula (IV), R⁴ is hydrogen, alkyl or substituted alkyl, more preferably R⁴ is hydrogen. In preferred embodiments, R³ is alkyl, more preferably methyl, substituted alkyl, aryl, more preferably phenyl, substituted aryl, preferably substituted phenyl, more preferably 4-substituted phenyl, still more preferably 4-fluorophenyl, or aralkyl. In preferred embodiments, R¹ and R² are each independently hydrogen, alkyl, substituted alkyl, or aralkyl. More preferably, R¹ is aralkyl and R² is alkyl, still more preferably R² is n-propyl.

In a specific embodiment of the present invention, the method of the present invention is conducted by administering to a mammal, in need thereof, a therapeutically effective amount of a compound of formula (IV) having the formula

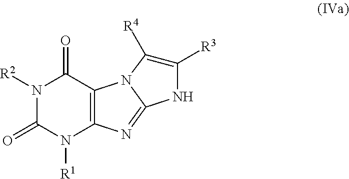

wherein R¹, R², R³ and R⁴ are as described above for compounds of formula (IV); or a pharmaceutically acceptable salt thereof.

Preferably, for compounds of formula (IVa), R⁴ is hydrogen, alkyl or substituted alkyl, more preferably R⁴ is hydrogen. In preferred embodiments, R³ is alkyl, more preferably methyl, substituted alkyl, aryl, more preferably phenyl, substituted aryl, preferably substituted phenyl, more preferably 4-substituted phenyl, still more preferably 4-fluorophenyl, or aralkyl. In preferred embodiments, R¹ and R² are each independently hydrogen, alkyl, substituted alkyl, or aralkyl. More preferably R² is alkyl, still more preferably propyl, and R¹ is aralkyl, more preferably benzyl.

In another specific embodiment of the present invention, the method of the present invention is conducted by administering to a mammal, in need thereof, a therapeutically effective amount of a compound of formula (IV) having the formula

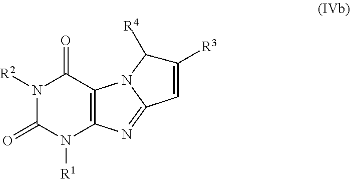

wherein R¹, R², R³ and R⁴ are as described above for compounds of formula (IV); or a pharmaceutically acceptable salt thereof.

Preferably, for compounds of formula (IVb), R⁴ is hydrogen, alkyl or substituted alkyl. In preferred embodiments, R³ is alkyl, substituted alkyl, aryl, more preferably phenyl, substituted aryl, preferably substituted phenyl, more preferably 4-substituted phenyl, still more preferably 4-fluorophenyl, or aralkyl. In preferred embodiments, R¹ and R² are each independently hydrogen, alkyl, substituted alkyl, or aralkyl. More preferably, R¹ is alkyl, still more preferably propyl, and R² is aralkyl, more preferably benzyl.

Non-limiting examples of compounds of formulae (IVa) and (IVb) include those listed herein below:
1-Benzyl-7-phenyl-3-propyl-1H-pyrrolo[1,2-f]purine-2,4(3H,6H)-dione;
1-Benzyl-7-phenyl-3-propyl-1H-imidazo[1,2-f]purine-2,4(3H,8H)-dione;
1-Benzyl-7-(4-methoxyphenyl)-3-propyl-1H-imidazo[1,2-f]purine-2,4(3H,8H)-dione;
1-Benzyl-7-(biphenyl-4-yl)-3-propyl-1H-imidazo[1,2-f]purine-2,4(3H,8H)-dione;
1-Benzyl-7-(4-fluorophenyl)-3-propyl-1H-imidazo[1,2-f]purine-2,4(3H,8H)-dione;
7-Phenyl-1,3-dipropyl-1H-imidazo[1,2-f]purine-2,4(3H,8H)-dione;
1,3-Diisobutyl-7-phenyl-1H-imidazo[1,2-f]purine-2,4(3H,8H)-dione;
1-Benzyl-7-methyl-3-propyl-1H-imidazo[1,2-f]purine-2,4(3H,8H)-dione;
1,3-Dimethyl-7-phenyl-1H-imidazo[1,2-f]purine-2,4(3H,8H)-dione;
7-(Biphenyl-4-yl)-1,3-dimethyl-1H-imidazo[1,2-f]purine-2,4(3H,8H)-dione;
7-(4-Chlorophenyl)-1,3-dimethyl-1H-imidazo[1,2-f]purine-2,4(3H,8H)-dione;
7-(4-Bromophenyl)-1,3-dimethyl-1H-imidazo[1,2-f]purine-2,4(3H,8H)-dione;
7-(4-Fluorophenyl)-1,3-dimethyl-1H-imidazo[1,2-f]purine-2,4(3H,8H)-dione;
7-(4-Methoxyphenyl)-1,3-dimethyl-1H-imidazo[1,2-f]purine-2,4(3H,8H)-dione;
1-Benzyl-7-methyl-3-propyl-1H-pyrrolo[1,2-f]purine-2,4(3H,6H)-dione;
1-Benzyl-7-ethyl-3-propyl-1H-pyrrolo[1,2-f]purine-2,4(3H,6H)-dione;
1-Benzyl-6,7-dimethyl-3-propyl-1H-pyrrolo[1,2-f]purine-2,4(3H,6H)-dione;
1-Benzyl-7-ethyl-3-propyl-1H-imidazo[1,2-f]purine-2,4(3H,8H)-dione;
1-Benzyl-7-isopropyl-3-propyl-1H-imidazo[1,2-f]purine-2,4(3H,8H)-dione;
1-Benzyl-7-t-butyl-3-propyl-1H-imidazo[1,2-f]purine-2,4(3H,8H)-dione;
1-Benzyl-7-cyclopropyl-3-propyl-1H-imidazo[1,2-f]purine-2,4(3H,8H)-dione;
1-Benzyl-7-cyclohexyl-3-propyl-1H-imidazo[1,2-f]purine-2,4(3H,8H)-dione;
1-Benzyl-6,7-dimethyl-3-propyl-1H-imidazo[1,2-f]purine-2,4(3H,8H)-dione;
1-Benzyl-7-ethyl-6-methyl-3-propyl-1H-imidazo[1,2-f]purine-2,4(3H,8H)-dione; and
1,3,7-Trimethyl-1H-imidazo[1,2-f]purine-2,4(3H,8H)-dione;
   or a pharmaceutically acceptable salt thereof.

Preferred are compounds of formula (IV) having the formula (IVa), especially preferred is the compound of the formula

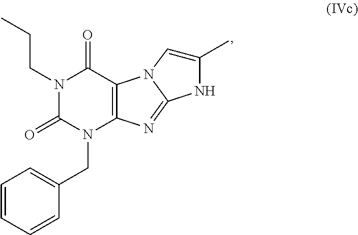

i.e., 1-benzyl-7-methyl-3-propyl-1H-imidazo[1,2-f]purine-2,4(3H,8H)-dione, or a pharmaceutically accepable salt thereof.

Yet in another specific embodiment of the present invention, the method of the present invention is conducted by administering to a mammal, in need thereof, a therapeutically effective amount of a compound of the formula

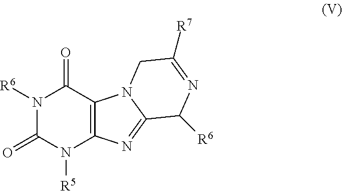

wherein

R⁵ and R⁶ are each independently hydrogen, alkyl, substituted alkyl, aralkyl, substituted aralkyl, alkenyl, substituted alkenyl, alkynyl, substituted alkynyl, aryl, or substituted aryl;

R⁷ is alkyl, substituted alkyl, aryl, substituted aryl, aralkyl, or substituted aralkyl; and

R⁸ is alkyl, substituted alkyl, aralkyl, substituted aralkyl, aryl, or substituted aryl;

or a pharmaceutically acceptable salt thereof.

Non-limiting examples of compounds of formula (V) include those listed herein below:
8-Benzyl-1-methyl-3-phenyl-6-propyl-1,4-dihydro-8H-1,2,4a,6,8,9-hexaaza-fluorene-5,7-dione; and
8-Benzyl-1-(2-hydroxy-ethyl)-3-phenyl-6-propyl-1,4-dihydro-8H-1,2,4a,6,8,9-hexaaza-fluorene-5,7-dione;
   or a pharmaceutically acceptable salt thereof.

As noted herein above, the present invention further provides a combination therapy for the prevention and treatment of atherosclerosis, and the subsequent prevention of stroke and heart attack, comprising an adenosine A₃ receptor antagonist in combination with at least one other therapeutic agent selected from the group consisting of (1) an ACE inhibitor; (2) an angiotensin II receptor blocker; (3) a renin inhibitor; (4) a diuretic; (5) a calcium channel blocker (CCB); (6) a beta-blocker; (7) a platelet aggregation inhibitor; (8) a cholesterol absorption modulator; (9) a HMG-Co-A reductase inhibitor; (10) a high density lipoprotein (HDL) increasing compound; (11) an ACAT inhibitor; and (12) an adenosine A_{2B} receptor antagonist; or in each case, a pharmaceutically acceptable salt thereof.

As referred herein above, the adenosine A₃ antagonists to be employed in the combination therapy of the present invention may optionally exhibit antagonistic activity on the other adenosine receptor subtypes, in particular, on the adenosine A_{2B} receptor subtype.

Inhibitors of the renin angiotensin system (RAS) are well known drugs that lower blood pressure and exert beneficial actions in hypertension and in congestive heart failure as described, e.g., in N. Eng. J. Med., 316: 1429-1435, 1987. The natural enzyme renin is released from the kidneys and cleaves angiotensinogen in the circulation to form the decapeptide angiotensin I. This is in turn cleaved by angiotensin converting enzyme (ACE) in the lungs, kidneys and other organs to form the octapeptide angiotensin II. The octapeptide increases blood pressure both directly by arterial vasoconstriction and indirectly by liberating from the adrenal glands the sodium-ion-retaining hormone aldosterone, accompanied by an increase in extracellular fluid volume. Inhibitors of the enzymatic activity of renin bring about a reduction in the formation of angiotensin I. As a result a smaller amount of angiotensin II is produced. The reduced concentration of that active peptide hormone is the direct cause of the antihypertensive effect of renin inhibitors.

Angiotensin II receptor blockers are understood to be those active agents that bind to the AT₁-receptor subtype of angiotensin II receptor but do not result in activation of the receptor. As a consequence of the blockade of the AT₁ receptor, these antagonists can be employed, e.g., as antihypertensive agents.

Suitable angiotensin II receptor blockers which may be employed in the combination of the present invention include AT₁ receptor antagonists having differing structural features, preferred are those with the non-peptidic structures. For example, mention may be made of the compounds that are selected from the group consisting of valsartan (U.S. Pat. No. 5,399,578; EP 443983), losartan (U.S. Pat. No. 5,138,069; EP 253310), candesartan (U.S. Pat. No. 5,703,110; U.S. Pat. No. 5,196,444; EP 459136), eprosartan (U.S. Pat. No. 5,185,351; EP 403159), irbesartan (U.S. Pat. No. 5,270,317; EP 454511), olmesartan (U.S. Pat. No. 5,616,599; EP 503785), tasosartan (U.S. Pat. No. 5,149,699; EP 539086), and telmisartan (U.S. Pat. No. 5,591,762; EP 502314).

Preferred AT₁-receptor antagonists are those agents that have reach the market, most preferred are losartan and valsartan or, in each case, a pharmaceutically acceptable salt thereof.

The interruption of the enzymatic degradation of angiotensin I to angiotensin II with ACE inhibitors is a successful variant for the regulation of blood pressure and, thus, also makes available a therapeutic method for the treatment of hypertension.

A suitable ACE inhibitor to be employed in the combination of the present invention is, e.g., a compound selected from the group consisting alacepril, benazepril, captopril, ceronapril, cilazapril, delapril, enalapril, fosinopril, imidapril, lisinopril, moexipril, moveltopril, perindopril, quinapril, ramipril, spirapril, temocapril, trandolapril and zofenopril, or in each case, a pharmaceutically acceptable salt thereof.

Preferred ACE inhibitors are those agents that have been marketed, most preferred ACE inhibitor is ramipril (U.S. Pat. No. 5,061,722).

Inhibitors of the enzymatic activity of renin bring about a reduction in the formation of angiotensin I. As a result a smaller amount of angiotensin II is produced. The reduced concentration of that active peptide hormone is the direct cause of, e.g., the hypotensive effect of renin inhibitors.

Suitable renin inhibitors include compounds having different structural features. For example, mention may be made of compounds which are selected from the group consisting of ditekiren, remikiren, terlakiren, and zankiren, preferably, in each case, the hydrochloride salt thereof.

In particular, the present invention relates to renin inhibitors disclosed in U.S. Pat. No. 5,559,111; No. 6,197,959 and No. 6,376,672, the entire contents of which are incorporated herein by reference.

Preferred renin inhibitors of the present invention include renin inhibitors disclosed in U.S. Pat. No. 6,197,959 and No. 6,376,672, in particular, RO 66-1132 and RO 66-1168 of formulae (VI) and (VII)

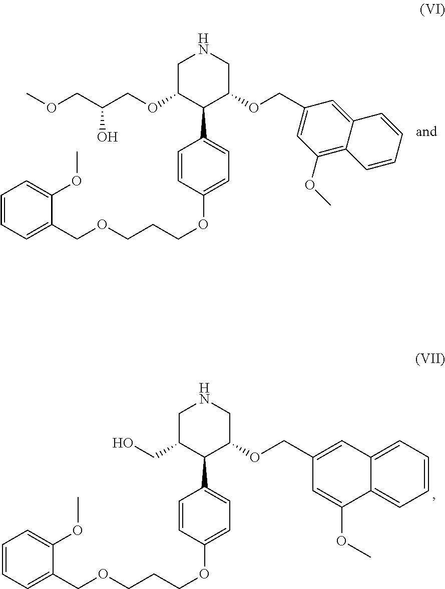

respectively, or in each case, a pharmaceutically acceptable salt thereof.

Preferred renin inhibitors also include δ-amino-γ-hydroxy-ω-aryl-alkanoic acid amide derivatives disclosed in U.S. Pat. No. 5,559,111, in particular, the compound of the formula

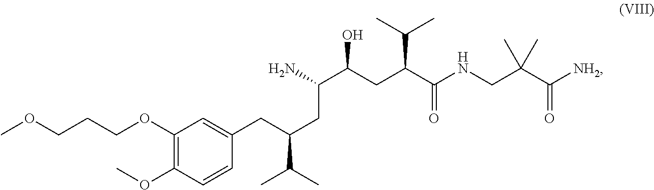

also known as aliskiren.

The term “aliskiren”, if not defined specifically, is to be understood both as the free base and as a salt thereof, especially a pharmaceutically acceptable salt thereof, most preferably a hemi-fumarate salt thereof.

A diuretic is, for example, a thiazide derivative selected from the group consisting of chlorothiazide, hydrochlorothiazide, methylclothiazide, and chlorothalidon. The most preferred diuretic is hydrochlorothiazide. A diuretic furthermore is a potassium sparing diuretic such as amiloride or triameterine, or a pharmaceutically acceptable salt thereof.

The class of CCBs essentially comprises dihydropyridines (DHPs) and non-DHPs, such as diltiazem-type and verapamil-type CCBs.

A CCB useful in said combination is preferably a DHP representative selected from the group consisting of amlodipine, felodipine, ryosidine, isradipine, lacidipine, nicardipine, nifedipine, niguldipine, niludipine, nimodipine, nisoldipine, nitrendipine and nivaldipine, and is preferably a non-DHP representative selected from the group consisting of flunarizine, prenylamine, diltiazem, fendiline, gallopamil, mibefradil, anipamil, tiapamil and verapamil, and in each case, a pharmaceutically acceptable salt thereof. All these CCBs are therapeutically used, e.g., as anti-hypertensive, anti-angina pectoris or anti-arrhythmic drugs.

Preferred CCBs comprise amlodipine, diltiazem, isradipine, nicardipine, nifedipine, nimodipine, nisoldipine, nitrendipine and verapamil or, e.g., dependent on the specific CCB, a pharmaceutically acceptable salt thereof. Especially preferred as DHP is amlodipine, or a pharmaceutically acceptable salt thereof, especially the besylate salt thereof. An especially preferred representative of non-DHPs is verapamil, or a pharmaceutically acceptable salt thereof, especially the hydrochloride salt thereof.

Beta-blockers suitable for use in the present invention include beta-adrenergic blocking agents (beta-blockers) which compete with epinephrine for beta-adrenergic receptors and interfere with the action of epinephrine. Preferably, the beta-blockers are selective for the beta-adrenergic receptor as compared to the alpha-adrenergic receptors, and so do not have a significant alpha-blocking effect. Suitable beta-blockers include compounds selected from acebutolol, atenolol, betaxolol, bisoprolol, carteolol, carvedilol, esmolol, labetalol, metoprolol, nadolol, oxprenolol, penbutolol, pindolol, propranolol, sotalol and timolol. Where the beta-blocker is an acid or base or otherwise capable of forming pharmaceutically acceptable salts or prodrugs, these forms are considered to be encompassed herein, and it is understood that the compounds may be administered in free form or in the form of a pharmaceutically acceptable salt or a prodrug, such as a physiologically hydrolizable and acceptable ester. For example, metoprolol is suitably administered as its tartrate salt, propranolol is suitably administered as the hydrochloride salt, and so forth.

Platelet aggregation inhibitors include, e.g., PLAVIX® (clopidogrel bisulfate), PLETAL® (cilostazol) and aspirin.

Cholesterol absorption modulators include, e.g., ZETIA® (ezetimibe).

HMG-Co-A reductase inhibitors (also called β-hydroxy-↑-methylglutaryl-co-enzyme-A reductase inhibitors or statins) are understood to be those active agents which may be used to lower lipid levels including plasma cholesterol levels.

HMG-Co-A reductase inhibitors include compounds having differing structural features. For example, mention may be made of the compounds which are selected from the group consisting of atorvastatin, cerivastatin, fluvastatin, lovastatin, pitavastatin, pravastatin, rosuvastatin and simvastatin, or in each case, a pharmaceutically acceptable salt thereof.

Preferred HMG-Co-A reductase inhibitors are those agents which have been marketed, most preferred are atorvastatin, rosuvastatin and simvastatin, or in each case, a pharmaceutically acceptable salt thereof.

HDL increasing compounds include, but are not limited to, cholesterol ester transfer protein (CETP) inhibitors. Examples of CETP inhibitors include those disclosed in U.S. Pat. No. 6,140,343 and No. 6,197,786, e.g., a compound known as torcetrapib; those disclosed in International PCT Application No. WO 2006014413, e.g., a compound known as anacetrapib; and those disclosed in U.S. Pat. No. 6,426,365, e.g., a compound known as JTT-705.

Acyl-CoA;cholesterol O-acyltransferase (ACAT) is an enzyme that catalyzes the synthesis of cholesterol ester from cholesterol, and plays a vital role in metabolism of cholesterol and absorption thereof in digestive organs and, therefore, inhibitors of the ACAT enzyme may be employed as anti-hyperlipidemic agents. Examples of ACAT inhibitors include, but are not limited to, avasimibe and pactimibe.

Adenosine A_{2B} receptor antagonists include, but are not limited to, PSB 1115 potassium salt, PSB 603, MRS 1754 and alloxazine (commercially available from Sigma-Aldrich and/or Tocris Bioscience). Other suitable antagonists include those disclosed in U.S. Pat. No. 6,545,002; U.S. Pat. No. 6,825,349; U.S. Pat. No. 6,916,804; U.S. Pat. No. 7,160,892; U.S. Pat. No. 7,205,403; and U.S. Pat. No. 7,342,006; e.g., a compound known as MRE-2029F20.

Preferably, a combination according to the present invention comprises an adenosine A₃ receptor antagonist and an angiotensin II antagonist, e.g., losartan or valsartan, or in each case, a pharmaceutically acceptable salt thereof, and optionally, a diuretic, e.g., hydrochlorothiazide, or a pharmaceutically acceptable salt thereof, and/or a HMG-Co-A reductase inhibitor, e.g., atorvastatin, rosuvastatin or simvastatin, or in each case, a pharmaceutically acceptable salt thereof.

Preferred is also a combination according to the present invention which comprises an adenosine A₃ receptor antagonist and an ACE inhibitor, e.g., ramipril, or a pharmaceutically acceptable salt thereof, and optionally, a diuretic, e.g., hydrochlorothiazide, or a pharmaceutically acceptable salt thereof, and/or a HMG-Co-A reductase inhibitor, e.g., atorvastatin, rosuvastatin or simvastatin, or in each case, a pharmaceutically acceptable salt thereof.

Preferred is also a combination according to the present invention which comprises an adenosine A₃ receptor antagonist and a renin inhibitor, e.g., aliskiren, or a pharmaceutically acceptable salt thereof, preferably the hemi-fumarate salt thereof, and optionally, a diuretic, e.g., hydrochlorothiazide, or a pharmaceutically acceptable salt thereof, and/or a HMG-Co-A reductase inhibitor, e.g., atorvastatin, rosuvastatin or simvastatin, or in each case, a pharmaceutically acceptable salt thereof.

Preferred is also a combination according to the present invention which comprises an adenosine A₃ receptor antagonist and a CCB, e.g., amlodipine, or a pharmaceutically acceptable salt thereof, and optionally, a diuretic, e.g., hydrochlorothiazide, or a pharmaceutically acceptable salt thereof, and/or a HMG-Co-A reductase inhibitor, e.g., atorvastatin, rosuvastatin or simvastatin, or in each case, a pharmaceutically acceptable salt thereof.

Preferred is also a combination according to the present invention which comprises an adenosine A₃ receptor antagonist and a beta-blocker, e.g., acebutolol, atenolol, betaxolol, bisoprolol, carteolol, carvedilol, esmolol, labetalol, metoprolol, nadolol, oxprenolol, penbutolol, pindolol, propranolol, sotalol and timolol, or a pharmaceutically acceptable salt thereof, and optionally, a diuretic, e.g., hydrochlorothiazide, or a pharmaceutically acceptable salt thereof, and/or a HMG-Co-A reductase inhibitor, e.g., atorvastatin, rosuvastatin or simvastatin, or in each case, a pharmaceutically acceptable salt thereof.

Preferred is also a combination according to the present invention which comprises an adenosine A₃ receptor antagonist and a platelet aggregation inhibitor, e.g., clopidogrel or aspirin, or a pharmaceutically acceptable salt thereof, and optionally, a diuretic, e.g., hydrochlorothiazide, or a pharmaceutically acceptable salt thereof, and/or a HMG-Co-A reductase inhibitor, e.g., atorvastatin, rosuvastatin or simvastatin, or in each case, a pharmaceutically acceptable salt thereof.

Preferred is also a combination according to the present invention which comprises an adenosine A₃ receptor antagonist and an adenosine A_{2B} receptor antagonist, e.g., MRE-2029F20, or a pharmaceutically acceptable salt thereof, and optionally, a diuretic, e.g., hydrochlorothiazide, or a pharmaceutically acceptable salt thereof, and/or a HMG-Co-A reductase inhibitor, e.g., atorvastatin, rosuvastatin or simvastatin, or in each case, a pharmaceutically acceptable salt thereof.

Preferred is also a combination according to the present invention which comprises an adenosine A₃ receptor antagonist and a diuretic, e.g., hydrochlorothiazide, or a pharmaceutically acceptable salt thereof, and optionally a HMG-Co-A reductase inhibitor, e.g., atorvastatin, rosuvastatin or simvastatin, or in each case, a pharmaceutically acceptable salt thereof.

Preferred is also a combination according to the present invention which comprises an adenosine A₃ receptor antagonist and a HMG-Co-A reductase inhibitor, e.g., atorvastatin, rosuvastatin or simvastatin, or in each case, a pharmaceutically acceptable salt thereof.

The structure of the active agents identified by generic or tradenames may be taken from the actual edition of the standard compendium “The Merck Index” or the Physician's Desk Reference or from databases, e.g. Patents International (e.g. IMS World Publications) or Current Drugs. The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

As referred to herein above, the adenosine A₃ receptor antagonists of the present invention, and the combination partners thereof, may be present as their pharmaceutically acceptable salts. If these compounds have, e.g., at least one basic center such as an amino group, they can form acid addition salts thereof. Similarly, the compounds having at least one acid group (for example COOH) can form salts with bases. Corresponding internal salts may furthermore be formed, if a compound comprises, e.g., both a carboxy and an amino group.

The corresponding active ingredients or a pharmaceutically acceptable salts may also be used in form of a solvate, such as a hydrate or including other solvents used, e.g., in their crystallization.

In yet another aspect, the present invention relates to pharmaceutical compositions comprising an adenosine A₃ receptor antagonist, or a pharmaceutically acceptable salt thereof, and a pharmaceutically acceptable carrier, for the inhibition of foam cell formation and, thus, the prevention and treatment of atherosclerosis, and the subsequent prevention of stroke and heart attack.

As referred herein above, the adenosine A₃ antagonists to be employed in the pharmaceutical compositions of the present invention may optionally exhibit antagonistic activity on the other adenosine receptor subtypes, in particular, on the adenosine A_{2B} receptor subtype.

Furthermore, the present invention provides pharmaceutical compositions comprising a therapeutically effective amount of a combination of an adenosine A₃ receptor antagonist and at least one other therapeutic agent selected from the group consisting of:
(1) an ACE inhibitor, preferably ramipril, a pharmaceutically acceptable salt thereof;
(2) an angiotensin II receptor blocker, preferably losartan or valsartan, or in each case, a pharmaceutically acceptable salt thereof;
(3) a renin inhibitor, preferably aliskiren, or a pharmaceutically acceptable salt thereof, e.g., the hemi-fumarate salt thereof;
(4) a diuretic, preferably hydrochlorothiazide, or a pharmaceutically acceptable salt thereof;
(5) a calcium channel blocker (CCB), preferably amlodipine, or a pharmaceutically acceptable salt thereof;
(6) a beta-blocker, or a pharmaceutically acceptable salt thereof;
(7) a platelet aggregation inhibitor, or a pharmaceutically acceptable salt thereof;
(8) a cholesterol absorption modulator, or a pharmaceutically acceptable salt thereof;
(9) a HMG-Co-A reductase inhibitor, preferably atorvastatin, rosuvastatin or simvastatin, or in each case, a pharmaceutically acceptable salt thereof;
(10) a high density lipoprotein (HDL) increasing compound, or a pharmaceutically acceptable salt thereof;
(11) an ACAT inhibitor, or a pharmaceutically acceptable salt thereof; and
(12) an adenosine A_{2B} receptor antagonist; or a pharmaceutically acceptable salt thereof;
   and a pharmaceutically acceptable carrier; for the prevention and treatment of atherosclerosis, e.g., slowing the progression and ultimate regression of atherosclerotic plaque, and the subsequent prevention of stroke and heart attack.

As disclosed herein above, an adenosine A₃ receptor antagonist may be co-administered as a pharmaceutical composition in combination with at least one other therapeutic agent selected from the group consisting of: (1) an ACE inhibitor, e.g., ramipril; (2) an angiotensin II receptor blocker, e.g., losartan or valsartan; (3) a renin inhibitor, e.g., aliskiren; (4) a diuretic, e.g., hydrochlorothiazide; (5) a calcium channel blocker (CCB), e.g., amlodipine; (6) a beta-blocker, e.g., metoprolol; (7) a platelet aggregation inhibitor; (8) a cholesterol absorption modulator; (9) a HMG-Co-A reductase inhibitor, e.g., atorvastatin, rosuvastatin or simvastatin; (10) a high density lipoprotein (HDL) increasing compound; (11) an ACAT inhibitor; and (12) an adenosine A_{2B} receptor antagonist; or in each case, a pharmaceutically acceptable salt thereof. The components may be administered together in any conventional dosage form, usually also together with a pharmaceutically acceptable carrier or diluent.

In carrying out the method of the present invention, the adenosine A₃ receptor antagonists of the present invention, or the combination partners thereof, may be formulated into pharmaceutical compositions suitable for administration via a variety of routes, such as oral or rectal, transdermal and parenteral administration to mammals, including man. For oral administration the pharmaceutical composition comprising an adenosine A₃ receptor antagonist, or a combination partner thereof, can take the form of solutions, suspensions, tablets, pills, capsules, powders, microemulsions, unit dose packets and the like. Preferred are tablets and gelatin capsules comprising the active ingredient together with: a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbants, colorants, flavors and sweeteners. Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-90%, preferably about 1-80%, of the active ingredient.

The amount of the compounds of the present invention required to be therapeutically effective will, of course, vary with the individual mammal being treated and is ultimately at the discretion of the medical or veterinary practitioner. The factors to be considered include the severity of condition being treated, the route of administration, the nature of the formulation, the mammal's body weight, surface area, age and general condition, and the particular compound(s) to be administered. Suitable regimens can be selected by one skilled in the art by considering such factors and by following, e.g., dosages reported in the literature and recommended in the Physician's Desk Reference (58^{th} ed., 2004).

Preferred dosages for the active ingredients of the pharmaceutical combinations according to the present invention are therapeutically effective dosages, especially those which are commercially available.

Normally, in the case of oral administration, an approximate daily dose from about 1 μg to about 3 g is to be estimated, e.g., for a patient of approximately 75 kg in weight.

For example, a suitable therapeutically effective dose of an adenosine A₃ receptor antagonist ranges from about 0.01 mg/kg to 100 mg/kg, preferably less than about 10 mg/kg, more preferably less than about 5 mg/kg, more preferably less than about 1 mg/kg, more preferably less than about 0.5 mg/kg/day, and most preferably less than about 0.1 mg/kg of the patient's body weight per day. In certain embodiments, the adenosine A₃ receptor antagonist is administered at a dosage of at least 0.01 mg/kg/day, about 0.05 mg/kg/day, about 0.1 mg/kg/day, about 0.5 mg/kg/day, about 1.0 mg/kg/day, or about 10 mg/kg/day.

In case of ACE inhibitors, preferred unit dosage forms of ACE inhibitors are, e.g., tablets or capsules comprising, e.g., from about 5 mg to about 20 mg, preferably 5 mg, 10 mg, 20 mg or 40 mg, of benazepril; from about 6.5 mg to 100 mg, preferably 6.25 mg, 12.5 mg, 25 mg, 50 mg, 75 mg or 100 mg, of captopril; from about 2.5 mg to about 20 mg, preferably 2.5 mg, 5 mg, 10 mg or 20 mg, of enalapril; from about 10 mg to about 20 mg, preferably 10 mg or 20 mg, of fosinopril; from about 2.5 mg to about 4 mg, preferably 2 mg or 4 mg, of perindopril; from about 5 mg to about 20 mg, preferably 5 mg, 10 mg or 20 mg, of quinapril; or from about 1.25 mg to about 5 mg, preferably 1.25 mg, 2.5 mg, or 5 mg, of ramipril. Preferred is once a day administration.

Angiotensin II receptor blockers, e.g., valsartan, are supplied in the form of a suitable unit dosage form, e.g., a capsule or tablet, comprising a therapeutically effective amount of an angiotensin II receptor blocker, e.g., from about 20 to about 320 mg of valsartan. The administration of the active ingredient may occur up to three times a day, starting, e.g., with a daily dose of 20 mg or 40 mg of an angiotensin II receptor blocker, e.g., valsartan, increasing to 80 mg daily and further to 160 mg daily, and finally up to 320 mg daily. Preferably, an angiotensin II receptor blocker, e.g., valsartan, is applied once a day or twice a day employing a unit dose of 80 mg or 160 mg, respectively. The dosages may be taken, e.g., in the morning, at mid-day or in the evening.

In case of renin inhibitors, e.g., aliskiren, the doses to be administered to warm-blooded animals, including man, of approximately 75 kg body weight, especially the doses effective for the inhibition of renin activity, e.g., in lowering blood pressure, are from about 3 mg to about 3 g, preferably from about 10 mg to about 1 g, e.g., from 20 mg/person/day to 200 mg/person/day, divided preferably into 1 to 4 single doses which may, e.g., be of the same size. Usually, children receive about half of the adult dose. The dose necessary for each individual can be monitored, e.g., by measuring the serum concentration of the active ingredient, and adjusted to an optimum level. Single doses comprise, e.g., 75 mg, 150 mg or 300 mg per adult patient.

In case of diuretics, preferred unit dosage forms are, e.g., tablets or capsules comprising, e.g., from about 5 mg to about 50 mg, preferably from about 6.25 mg to about 25 mg. A daily dose of 6.25 mg, 12.5 mg or 25 mg of hydrochlorothiazide is preferably administered once a day.

In case of CCBs, e.g., amlodipine, preferred unit dosage forms are, e.g., tablets or capsules comprising, e.g., from about 1 mg to about 40 mg, preferably from 2.5 mg to 20 mg daily when administered orally.

In case of HMG-Co-A reductase inhibitors, preferred unit dosage forms of HMG-Co-A reductase inhibitors are, e.g., tablets or capsules comprising, e.g., from about 5 mg to about 120 mg, preferably, when using atorvastatin, e.g., 10 mg, 20 mg, 40 mg or 80 mg of atorvastatin, e.g., administered once a day.

In the case of adenosine A_{2B} receptor antagonists, preferred unit dosage forms are, e.g., tablets or capsules comprising, e.g., from about 5 mg to about 1 g, preferably from about 50 mg to about 100 mg, administered up to three times a day.

Since the present invention relates to methods for the prevention and treatment of atherosclerosis with a combination of compounds which may be administered separately, the invention also relates to combining separate pharmaceutical compositions in a kit form. The kit may comprise, e.g., two separate pharmaceutical compositions: (1) a composition comprising an adenosine A₃ receptor antagonist, or a pharmaceutically acceptable salt thereof, and a pharmaceutically acceptable carrier or diluent; and (2) a composition comprising at least one other therapeutic agent selected from the group consisting of an ACE inhibitor, an angiotensin II receptor blocker, a renin inhibitor, a diuretic, a calcium channel blocker (CCB), a beta-blocker, a platelet aggregation inhibitor, a cholesterol absorption modulator, a HMG-Co-A reductase inhibitor, a high density lipoprotein (HDL) increasing compound, an ACAT inhibitor, and an adenosine A_{2B} receptor antagonist, or in each case, a pharmaceutically acceptable salt thereof, and a pharmaceutically acceptable carrier or diluent. The amounts of (1) and (2) are such that, when co-administered separately a beneficial therapeutic effect(s) is achieved. The kit comprises a container for containing the separate compositions such as a divided bottle or a divided foil packet, wherein each compartment contains a plurality of dosage forms (e.g., tablets) comprising, e.g., (1) or (2). Alternatively, rather than separating the active ingredient-containing dosage forms, the kit may contain separate compartments each of which contains a whole dosage which in turn comprises separate dosage forms. An example of this type of kit is a blister pack wherein each individual blister contains two (or more) tablets, one (or more) tablet(s) comprising a pharmaceutical composition (1), and the second (or more) tablet(s) comprising a pharmaceutical composition (2). Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician. In the case of the instant invention a kit therefore comprises:

(1) a therapeutically effective amount of a composition comprising an adenosine A₃ receptor antagonist, or a pharmaceutically acceptable salt thereof, and a pharmaceutically acceptable carrier or diluent, in a first dosage form;
(2) a composition comprising at least one other therapeutic agent selected from the group consisting of an ACE inhibitor, an angiotensin II receptor blocker, a renin inhibitor, a diuretic, a calcium channel blocker (CCB), a beta-blocker, a platelet aggregation inhibitor, a cholesterol absorption modulator, a HMG-Co-A reductase inhibitor, a high density lipoprotein (HDL) increasing compound, ACAT inhibitor, and an adenosine A_{2B} receptor antagonist, or in each case, a pharmaceutically acceptable salt thereof, in an amount such that, following administration, a beneficial therapeutic effect(s) is achieved, and a pharmaceutically acceptable carrier or diluent, in a second dosage form; and
(3) a container for containing said first and second dosage forms.

The action of an adenosine A₃ receptor antagonist, alone or in combination with at least one other therapeutic agent selected from the group consisting of: (1) an ACE inhibitor; (2) an angiotensin II receptor blocker; (3) a renin inhibitor; (4) a diuretic; (5) a calcium channel blocker (CCB); (6) a beta-blocker; (7) a platelet aggregation inhibitor; (8) a cholesterol absorption modulator; (9) a HMG-Co-A reductase inhibitor; (10) a high density lipoprotein (HDL) increasing compound; (11) an ACAT inhibitor; and (12) an adenosine A_{2B} receptor antagonist; or in each case, a pharmaceutically acceptable salt thereof; may be demonstrated inter alia experimentally by means of in vitro and/or in vivo tests, e.g., as described herein in the illustrative Examples.

An adenosine A₃ receptor antagonist, or a pharmaceutical salt thereof, or the combination partners thereof, can be administered by various routes of administration. Each agent can be tested over a wide-range of dosages to determine the optimal drug level for each therapeutic agent alone, or in the specific combination thereof, to elicit the maximal response. For these studies, it is preferred to use treatment groups consisting of at least 6 animals per group. Each study is best performed in away wherein the effects of the combination treatment group are determined at the same time as the individual components are evaluated. Although drug effects may be observed with acute administration, it is preferable to observe responses in a chronic setting. The long-term study is of sufficient duration to allow for the full development of compensatory responses to occur and, therefore, the observed effect will most likely depict the actual responses of the test system representing sustained or persistent effects.

Representative studies may be carried out, e.g., by employing the WHHL (Watanable heritable hyperlipidemic) rabbit model for familial hypercholesterolemia (Atherosclerosis, 36: 261-268, 1980), or by employing an apolipoprotein E knockout mouse model which has now become one of the primary models for atherosclerosis (Arterioscler. Thromb. Vasc. Biol., 24: 1006-1014, 2004; Trends Cardiovasc. Med., 14: 187-190, 2004). The apolipoprotein E knockout mouse studies may be performed, e.g., as described by Johnson et al. in Circulation, 111: 1422-1430, 2005, or using modifications thereof.

The available results indicate that adenosine A₃ receptor antagonists may be employed for the inhibition of foam cell formation and, thus, the prevention and treatment of atherosclerosis, and the subsequent prevention of stroke and heart attack, independent of the antihypertensive effect of adenosine A₃ receptor antagonists. More surprisingly, it has been demonstrated that adenosine A₃ receptor antagonists may be employed for the regression of atherosclerotic plaque.

Furthermore, it has been found that, a combination of an adenosine A₃ receptor antagonist with at least one other therapeutic agent selected from the group consisting of: (1) an ACE inhibitor; (2) an angiotensin II receptor blocker; (3) a renin inhibitor; (4) a diuretic; (5) a calcium channel blocker (CCB); (6) a beta-blocker; (7) a platelet aggregation inhibitor; (8) a cholesterol absorption modulator; (9) a HMG-Co-A reductase inhibitor; (10) a high density lipoprotein (HDL) increasing compound; (11) an ACAT inhibitor; and (12) an adenosine A_{2B} receptor antagonist; or in each case, a pharmaceutically acceptable salt thereof; achieves greater therapeutic effect than the administration of the other therapeutic agents alone. Greater efficacy may also be documented as a prolonged duration of action. The duration of action can be monitored as either the time to return to baseline prior to the next dose or as the area under the curve (AUC).

Further benefits are that lower doses of the individual drugs to be combined according to the present invention can be used to reduce the dosage, e.g., that the dosages need not only often be smaller but are also applied less frequently, or can be used to diminish the incidence of side effects. The combined administration of an adenosine A₃ receptor antagonist with at least one other therapeutic agent selected from the group consisting of: (1) an ACE inhibitor; (2) an angiotensin II receptor blocker; (3) a renin inhibitor; (4) a diuretic; (5) a calcium channel blocker (CCB); (6) a beta-blocker; (7) a platelet aggregation inhibitor; (8) a cholesterol absorption modulator; (9) a HMG-Co-A reductase inhibitor; (10) a high density lipoprotein (HDL) increasing compound; (11) an ACAT inhibitor; and (12) an adenosine A_{2B} receptor antagonist; or in each case, a pharmaceutically acceptable salt thereof; results in a significant response in a greater percentage of treated patients, i.e., a greater responder rate results.

It can be shown that a combination therapy with an adenosine A₃ receptor antagonist and at least one other therapeutic agent selected from the group consisting of: (1) an ACE inhibitor; (2) an angiotensin II receptor blocker; (3) a renin inhibitor; (4) a diuretic; (5) a calcium channel blocker (CCB); (6) a beta-blocker; (7) a platelet aggregation inhibitor; (8) a cholesterol absorption modulator; (9) a HMG-Co-A reductase inhibitor; (10) a high density lipoprotein (HDL) increasing compound; (11) an ACAT inhibitor; and (12) an adenosine A_{2B} receptor antagonist; or in each case, a pharmaceutically acceptable salt thereof; results in a more effective therapy for the prevention and treatment of atherosclerosis, and the subsequent prevention of stroke and heart attack. In particular, all the more surprising is the finding that a combination of the present invention results in a beneficial, especially a synergistic, therapeutic effect but also in benefits resulting from combined treatment such as a surprising prolongation of efficacy.

The invention furthermore relates to the use of an adenosine A₃ receptor antagonist alone or in combination with at least one other therapeutic agent selected from the group consisting of: (1) an ACE inhibitor; (2) an angiotensin II receptor blocker; (3) a renin inhibitor; (4) a diuretic; (5) a calcium channel blocker (CCB); (6) a beta-blocker; (7) a platelet aggregation inhibitor; (8) a cholesterol absorption modulator; (9) a HMG-Co-A reductase inhibitor; (10) a high density lipoprotein (HDL) increasing compound; (11) an ACAT inhibitor; and (12) an adenosine A_{2B} receptor antagonist; or in each case, a pharmaceutically acceptable salt thereof; for the manufacture of a medicament for the prevention and treatment of atherosclerosis, and the subsequent prevention of stroke and heart attack.

Accordingly, another embodiment of the present invention relates to the use of an adenosine A₃ receptor antagonist alone or in combination with at least one other therapeutic agent selected from the group consisting of: (1) an ACE inhibitor, or a pharmaceutically acceptable salt thereof; (2) an angiotensin II receptor blocker, or a pharmaceutically acceptable salt thereof; (3) a renin inhibitor, or a pharmaceutically acceptable salt thereof; (4) a diuretic, or a pharmaceutically acceptable salt thereof; (5) a calcium channel blocker (CCB), or a pharmaceutically acceptable salt thereof; (6) a beta-blocker, or a pharmaceutically acceptable salt thereof; (7) a platelet aggregation inhibitor, or a pharmaceutically acceptable salt thereof; (8) a cholesterol absorption modulator, or a pharmaceutically acceptable salt thereof; (9) a HMG-Co-A reductase inhibitor, or a pharmaceutically acceptable salt thereof; (10) a high density lipoprotein (HDL) increasing compound; (11) an ACAT inhibitor; and (12) an adenosine A_{2B} receptor antagonist; or in each case, a pharmaceutically acceptable salt thereof; for the manufacture of a medicament for the prevention and treatment of atherosclerosis, and the subsequent prevention of stroke and heart attack.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative of certain aspects of the present invention and are not a limitation of the scope of the present invention in any way. The abbreviations used herein throughout the specification are those generally known in the art.

### Materials and Methods

### Cell Culture

The human myelomonocytic cell line U937 was obtained from ATCC and maintained in RPMI 1640 medium supplemented with 10% fetal calf serum, L-glutamine (2 mM), 100 U/mL penicillin, 100 μg/mL streptomycin, at 37° C. in 5% CO₂/95% air.

Preparation of Human Macrophages (HM) from Peripheral Blood

Peripheral blood mononuclear cells were isolated from buffy coats the Ficoll-Hypaque gradient (Ficoll-Paque, research Grade, Amersham Pharmacia Biotech AB, Cologno Monzese, Italy) as described previously by Gessi et al. (Mol. Pharmacol., 65: 711-719, 2004). Monocytes were selected by adhesion in RPMI 1640 medium containing 2 mM glutamine, 5% human AB serum (Sigma), 100 U/mL penicillin and 100 μg/mL streptomycin, and differentiated into macrophages by adhesion over 7 days.

### Hypoxic Treatment

Hypoxic exposures were done in a modular incubator chamber and flushed with a gas mixture containing 1% O₂, 5% CO₂ and balance N₂ (MiniGalaxy, RSBiotech, Irvine, Scotland).

### Foam Cell (FC) Formation

U937 cells were induced to differentiate into macrophages by treatment with phorbol myristate acetate (PMA, 40 nM) for 72 h. Before use oxLDL was dialyzed against 1 L of 0.15 M sodium chloride and 0.3 mM EDTA (pH 7.4) for 12 h at 4° C., then against RPMI 1640 medium (two changes, 1 L/each change) for 24 h. All dialyses were carried out with Pierce Slide-A-Lyzer cassettes (10,000 molecular wheight cut-off). After dialysis, lipoproteins were sterilized by passing them through a 0.45 μm (pore-size) filter, then added (50-100 μg/mL, Intracel, Frederick, Md.) to PMA-treated U937 cells and incubated in serum-free RPMI 1640 for 48 h. All treatments of cells with adenosine were carried out in the presence of adenosine deaminase (ADA) inhibitor, erythro-9-(2-hydroxy-3-nonyl)adenine (EHNA, 5 μM), and those with adenosine agonists were performed in the presence of ADA.

### Oil Red O-Stain Analysis

Treatment of PMA-differentiated U937 cells with test agents was performed 2 h before addition of oxLDL. After exposition to oxLDL under hypoxia for 24 h, U937 cells were fixed in phosphate buffered saline-buffered 4% paraformaldehyde solution for 15 min and then air dried. Oil red O (in 60% isopropanol) staining was done for 15 min essentially as described previously by Kalayoglu and Byrne (Infect. Immun., 66: 5067-5072, 1998). Cells were viewed under a bright-field microscope in 100× fields using a Nikon's Eclipse E800 microscope. Foam cells were defined as macrophages in which cytoplasm was filled with Oil red O-stainable lipid droplets.

### Real-Time RT-PCR

Total cytoplasmic RNA was extracted by the acid guanidinium thiocyanate phenol method. Quantitative real-time RT-PCR assay (Higuchi et al., Biotechnology, 11:1026-1030, 1993) of adenosine receptor mRNAs was carried out using gene-specific fluorescently labeled TaqMan MGB probe (minor groove binder) in a ABI Prism 7700 Sequence Detection System (Applied Biosystems, Warrington Cheshire, UK). For the real-time RT-PCR of A₁, A_{2A}, A_{2B} and A₃ adenosine subtypes the Assays-On-Demand™ Gene expression Products NM 000674, NM 000675, NM 000676 and NM 000677 were used, respectively. Moreover curves of adenosine receptors cDNA plasmid standards with a range spanning at least six orders of magnitude (10⁻¹¹-10⁻¹⁶ g/μL) were generated. These standard curves displayed a linear relationship between Ct values and the logarithm of plasmid amount (Gessi et al., Mol. Pharmacol., 67: 2137-2147, 2005). Quantification of adenosine receptor messages was made by interpolation from standard curve of Ct values generated from the plasmid dilution series (Kalayoglu and Byrne, Infect. Immun., 66: 5067-5072, 1998). For the real-time RT-PCR of HIF-1α, VEGF and IL-8 the Assays-On-Demand™ Gene expression Products NM, NM and NM were used, respectively. For the real-time RT-PCR of the reference gene the endogenous control human β-actin kit was used, and the probe was fluorescent-labeled with VIC™ (Applera).

### Membrane Preparation

U937 cells and macrophages were homogenized, respectively, in hypotonic buffer and phosphate-buffered saline (PBS), with a Polytron (Kinematica), and centrifuged for 30 min at 48,000×g as described previously (Gessi et al., Mol. Pharmacol., 65: 711-719, 2004). The protein concentration was determined according to a Bio Rad method (Bradford, Anal. Biochem., 72: 248-254, 1976) with bovine albumin as a standard reference.

### Binding Experiments

Binding assays were carried out according to Gessi et al. (Mol. Pharmacol., 65: 711-719, 2004). In saturation experiments, membranes (70 μg of protein per assay) were incubated with 50 mM Tris HCl buffer (10 mM MgCl₂ for A_{2A}; 10 mM MgCl₂, 1 mM EDTA and 0.1 mM benzamidine for A_{2B}; and 10 mM MgCl₂ and 1 mM EDTA for A₃) pH 7.4, and increasing concentrations of 1,3-dipropyl-8-cyclopentylxanthine ([³H]DPCPX) (0.4-40 nM); (4-(2-[7-amino-2-(2-furyl)-[1,2,4]triazolo-[2,32]-[1,3,6]-triazinyl-amino]ethyl)-phenol) ([³H]ZM 241385) (0.3-30 nM); N-benzo[1,3]dioxol-5-yl-2-[5-(1,3-dipropyl-2,6-dioxo-2,3,6,7-tetrahydro-1H-purin-8-yl)-1-methyl-1H-pyrazol-3-yl-oxy]-acetamide] ([³H]MRE 2029F20) (0.4-40 nM); 5-N-(4-methoxyphenyl-carbamoyl)amino-8-propyl-2-(2furyl)-pyrazolo-[4,3e]-1,2,4-triazolo[1,5-c]pyrimidine ([³H]MRE 3008F20) (0.4-40 nM) to label A₁, A_{2A}, A_{2B} and A₃ adenosine receptors, respectively. The filter bound radioactivity was counted on Top Count Microplate Scintillation Counter (efficiency 57%) with Micro-Scint 20.

### Western Blot Analysis

Whole cell lysates were prepared as described previously (27). Adenosine receptors were evaluated by using specific antibodies towards human adenosine A₁, A_{2A}, A_{2B} (Alpha Diagnostic) and A₃ receptors (Aviva) (1:1000 dilution). In experiments aimed to detect HIF, western blot analyses were performed using antibody against HIF-1a (1:250 dilution) and HIF-1β (1:1000 dilution) in 5% non-fat dry milk in PBS/0.1% Tween-20 overnight at 4° C. The protein concentration was determined using BCA protein assay kit (Pierce, Rockford, Ill.). Tubulin (1:250) was used to ensure equal protein loading. Immunoreactivity was assessed and quantified by using a VersaDoc Imaging System (Bio-Rad).

### Enzyme-Linked Immunosorbent Assay (ELISA)

The levels of VEGF and IL-8 protein secreted by the cells in the medium were determined by VEGF and IL-8 ELISA kits (R&D Systems) according to the manufacturer's instructions. The data were presented as mean±SD from three independent experiments.

Treatment of Cells with siRNA

Foam cells were plated in six-well plates and grown to 50-70% confluence before transfection. Transfection of siRNA was performed at a concentration of 100 nM using RNAiFect™ Transfection Kit (Qiagen). A non-specific control ribonucleotide sense strand (5′-ACU CUA UCU GCA CGC UGA CdTdT-3′) and antisense strand (5′-dTdT UGA GAU AGA CGU GCG ACU G-3′) were used under identical conditions as already reported by Merighi et al. (Neoplasia, 7: 894-903, 2005). The A₁, A_{2A}, A_{2B}, A₃AR and HIF-1α siRNAs were obtained from Santa Cruz Biotechnology (Santa Cruz, Calif.).

### Statistical Analysis

All values in the figures and text are expressed as mean±standard error (S.E.) of N observation (with N≧3). Data sets were examined by analysis of variance (ANOVA) and Dunnett's test (when required). A P-value less than 0.05 was considered statistically significant.

### Results

Expression of Adenosine Receptors mRNA in PMA-Treated U937, Macrophages and U937-Derived Foam Cells Under Normoxic and Hypoxic Conditions

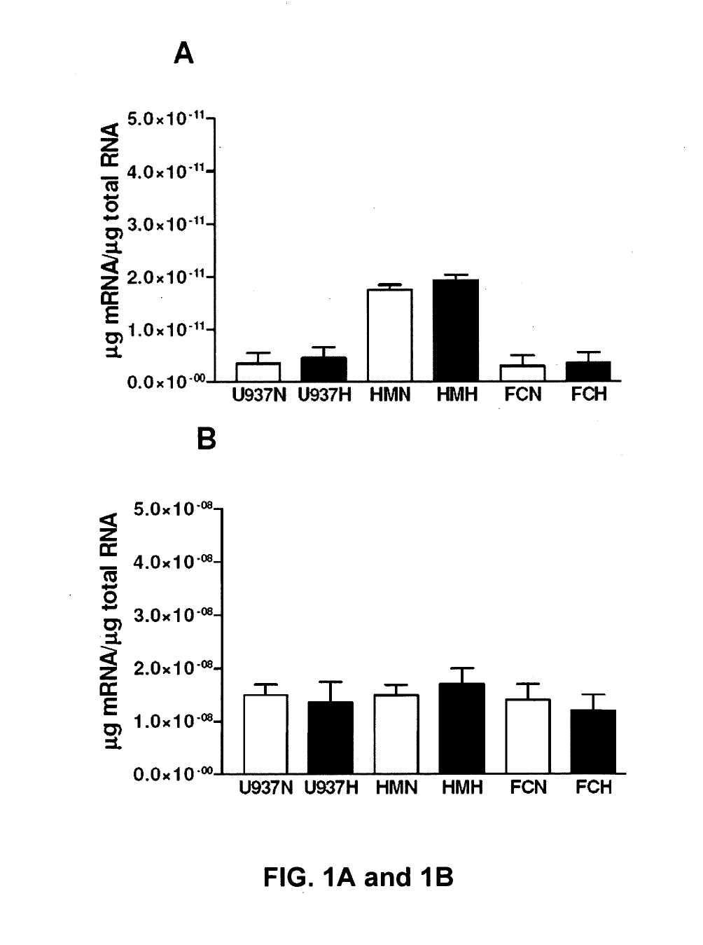

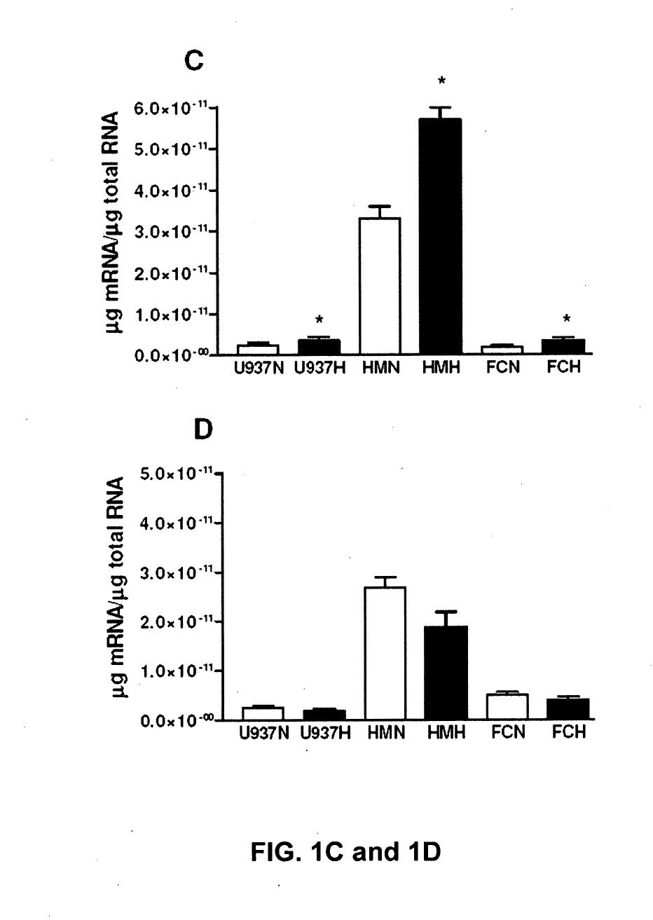

Expression of adenosine receptors mRNA was evaluated through real-time RT-PCR experiments in PMA-treated U937, human macrophages and U937-derived foam cells in normoxic and hypoxic conditions. As for the A₁ subtype it was expressed at similar levels in all three cellular models both in normoxia and hypoxia (1.3±0.2, 1.1±0.1, 1.2±0.1 fold of increase in normoxic vs. hypoxic U937, human macrophages and foam cells, respectively, FIG. 1A). Likewise, the A_{2A} and A₃ receptor subtypes were expressed at similar levels in all three cell types investigated both in normoxia and hypoxia (A_{2A} 0.9±0.1, 1.1±0.2, 0.9±0.1; and A₃ 0.7±0.1, 0.7±0.1, 0.8±0.1; fold of increase in normoxic vs. hypoxic U937, human macrophages and foam cells, respectively, FIGS. 1B and 1D). The A_{2B} receptor subtype expression was at the highest levels in human macrophages and was significantly elevated by hypoxia in all three cell types (A_{2B} 1.5±0.2, 1.8±0.1, 1.9±0.1 fold of increase in normoxic vs. hypoxic U937, macrophages and foam cells, respectively, FIG. 1C).

Evaluation of adenosine receptors message was made by interpolation from standard curve of Ct values generated from the plasmid dilution series. Analogue results were obtained when the expression level of adenosine receptors was normalized to the expression level of β-actin.

### Expression of Adenosine Receptors Protein in PMA-Treated U937 Cells, Human Macrophages and U937-Derived Foam Cells by Means of Western Blotting and Binding Experiments

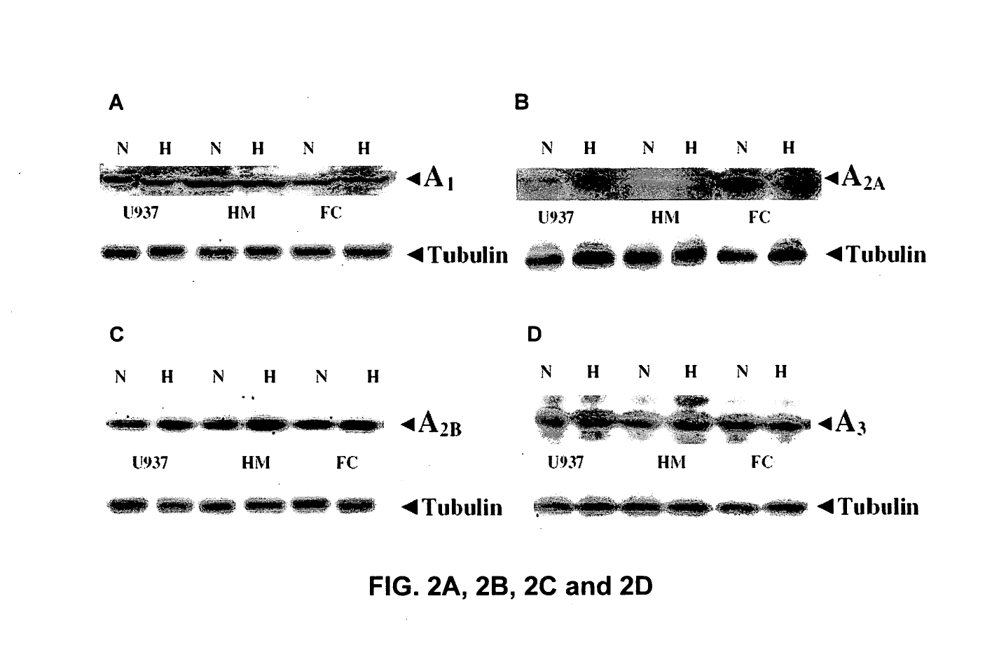

The protein evaluation of all adenosine receptor subtypes was examined, through western blotting experiments, in PMA-treated U937, human macrophages and U937-derived foam cells in normoxic and hypoxic conditions. The presence of all adenosine receptors was observed in all three cell types investigated according to mRNA data, as reported in FIG. 2.

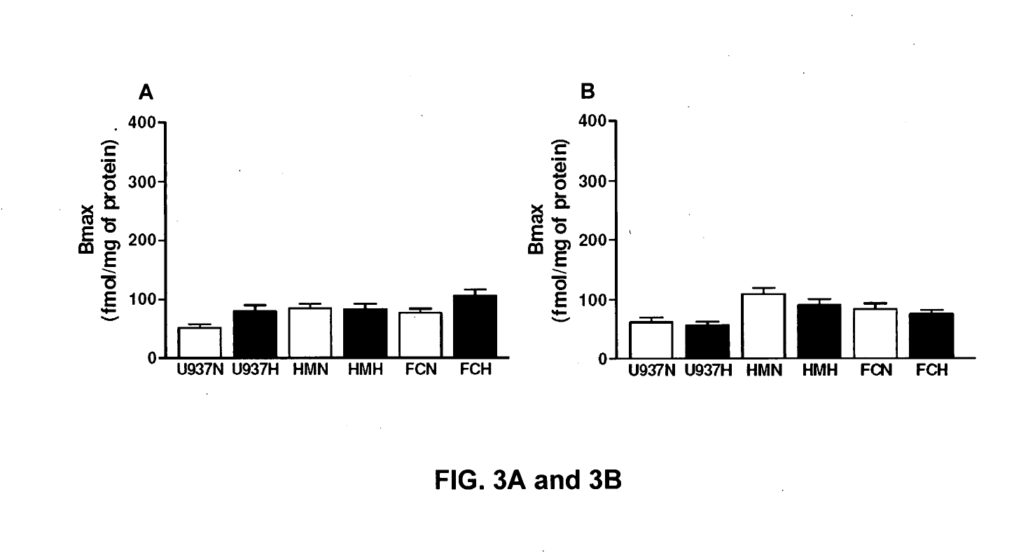

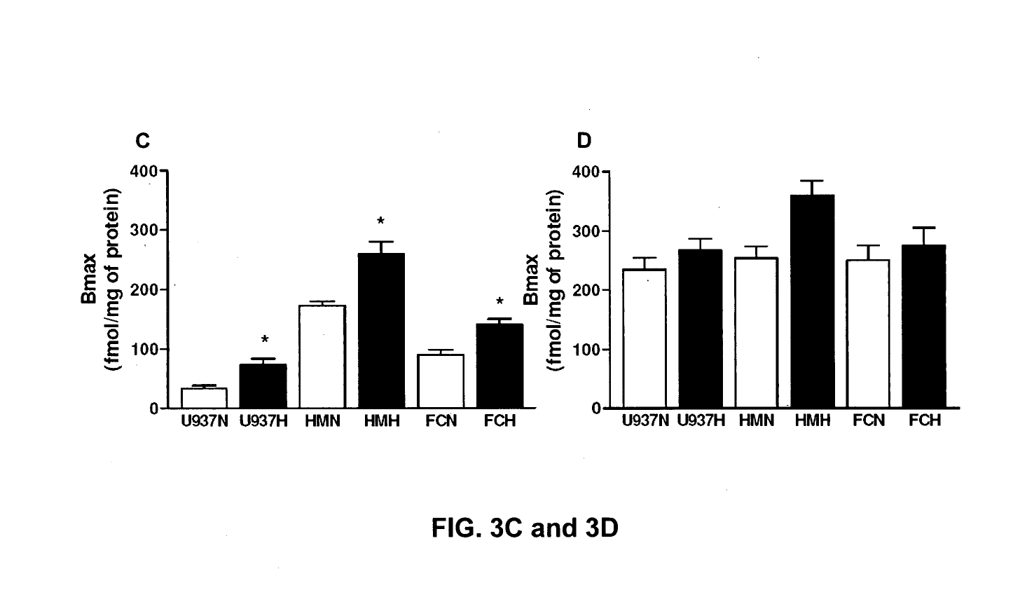

In order to quantify the amount of protein of the different adenosine subtypes we performed binding studies. [³H]DPCPX, [³H]ZM 241385, [³H]MRE-2029F20 and [³H]MRE-3008F20 antagonist radioligands were used in order to evaluate affinity (K_{D}, nM) and density (Bmax, fmol/mg of protein) values of A₁, A_{2A}, A_{2B} and A₃ receptors, respectively. As for A₁ receptors in U937 cells K_{D} values were 4.0±0.3 and 4.4±0.4, and Bmax values were 52±6, 80±10 fmol/mg of protein, respectively, in normoxic and hypoxic conditions; in human macrophages K_{D} values were of 2.8±0.3 and 2.8±0.4, and Bmax values were 85±9 and 83±10, respectively, in normoxic and hypoxic conditions; in foam cells K_{D} values were 3.3±0.5 and 3.7±0.6, and Bmax values were 78±10 and 102±12, respectively, in normoxic and hypoxic conditions (FIG. 3A). As for A₂,8, receptors in U937 cells K_{D} values were 2.8±0.3 and 2.5±0.2, and Bmax values were 62±9 and 57±8, respectively, in normoxic and hypoxic conditions; in human macrophages K_{D} values were of 2.2±0.3 and 2.3±0.3, and Bmax values were 109±12 and 90±10, respectively, in normoxic and hypoxic conditions; in foam cells K_{D} values were 2.1±0.1 and 2.2±0.1, and Bmax values were 84±9 and 75±7, respectively, in normoxic and hypoxic conditions (FIG. 3B). As for A_{2B} receptors in U937 cells K_{D} values were 4.3±0.4 and 4.1±0.5, and Bmax values were 33±3 and 73±6, respectively, in normoxic and hypoxic conditions; in human macrophages K_{D} values were of 4.9±0.3 and 4.8±0.6, and Bmax values were 173±15 and 240±18, respectively in normoxic and hypoxic conditions; in foam cells K_{D} values were 2.0±0.2 and 1.98±0.2, and Bmax values were 90±8 and 140±12, respectively, in normoxic and hypoxic conditions (FIG. 3C). Finally, as for A₃ receptors in U937 cells K_{D} values were 1.5±0.1 and 2.0±0.1, and Bmax values were 235±26 and 267±28, respectively in normoxic and hypoxic conditions; in human macrophages K_{D} values were of 4.5±0.5 and 4.8±0.7, and Bmax values were 254±24 and 360±33, respectively in normoxic and hypoxic conditions; in foam cells K_{D} values were 1.7±0.1 and 2.3±0.1, and Bmax values were 250±30 and 275±32, fmol/mg of protein, respectively, in normoxic and hypoxic conditions (FIG. 3D).

### Adenosine Receptors Induce HIF-1α Protein Accumulation in Hypoxia

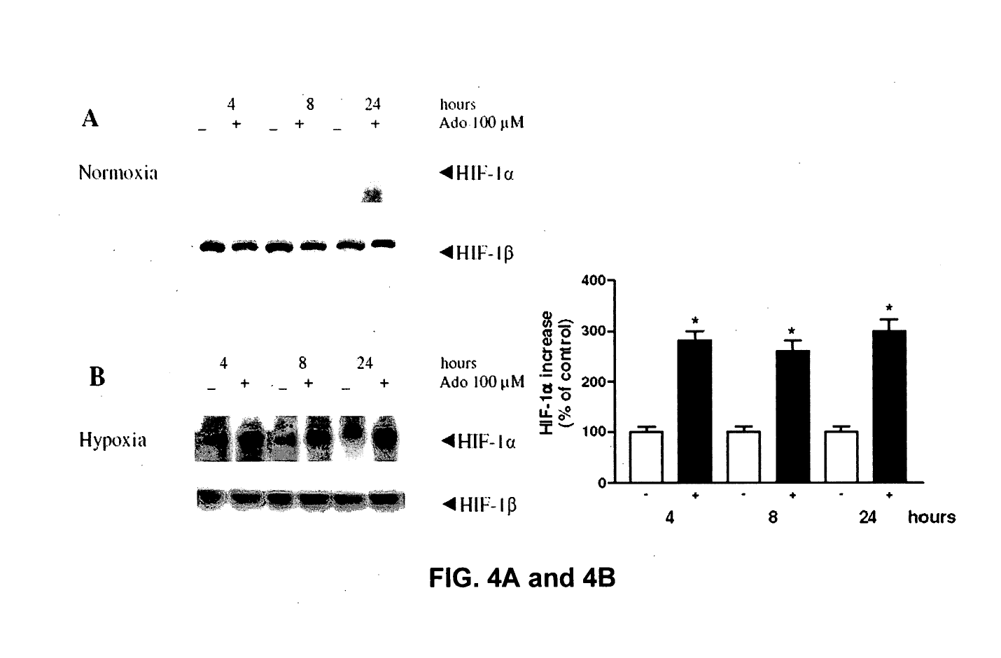

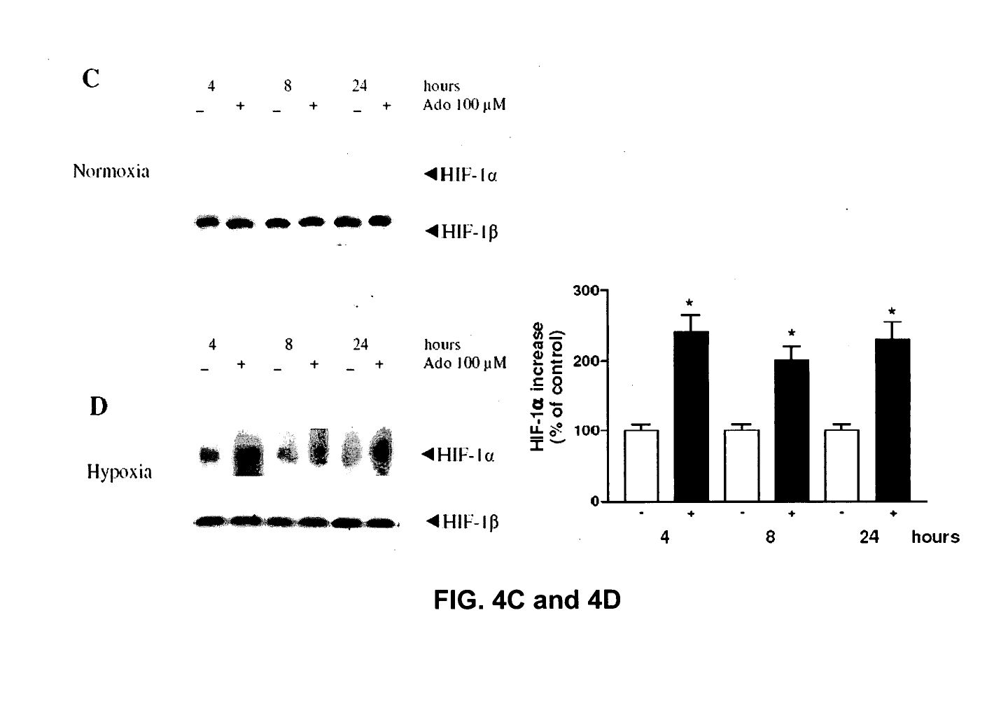

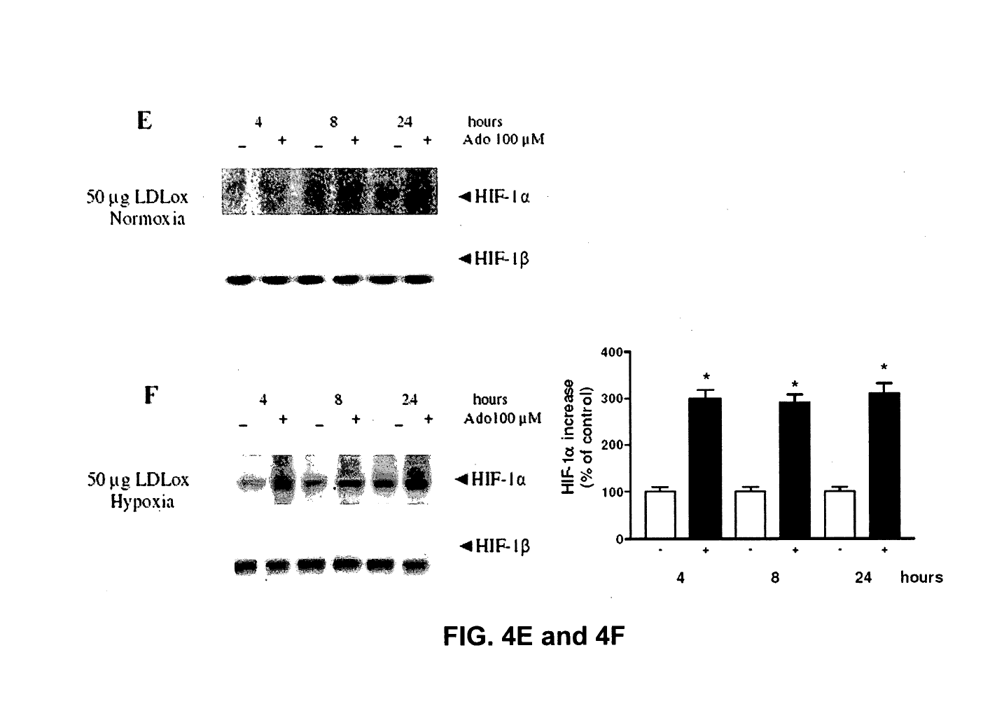

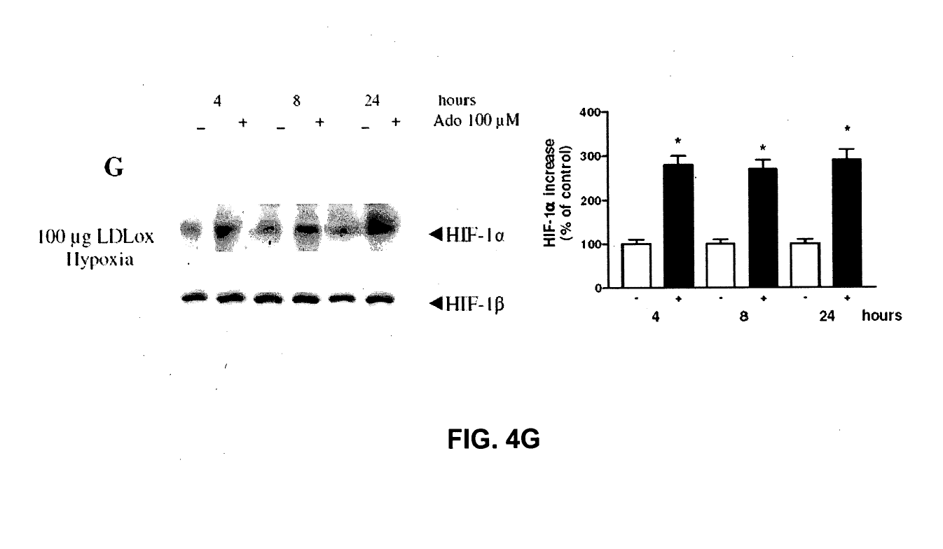

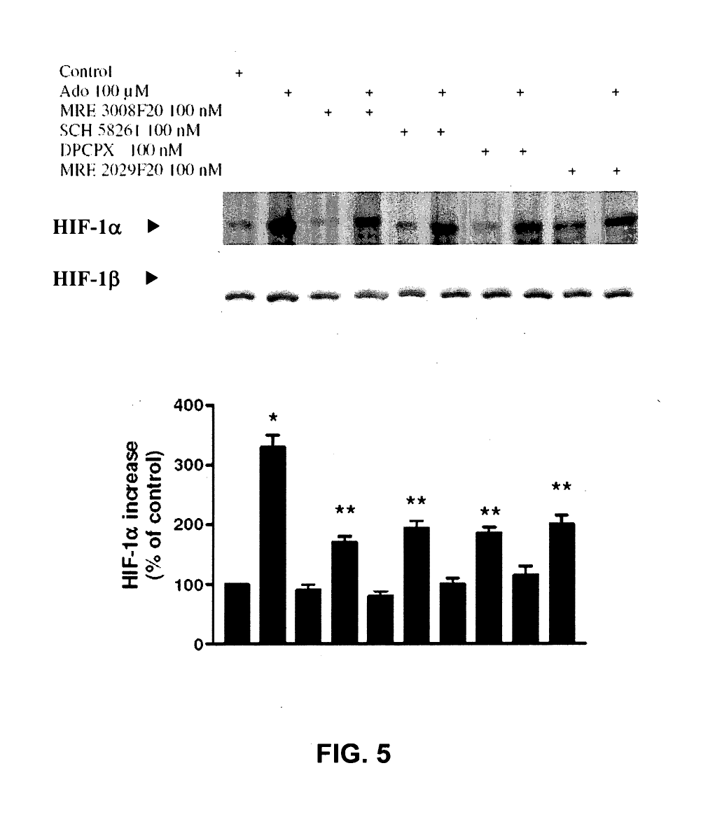

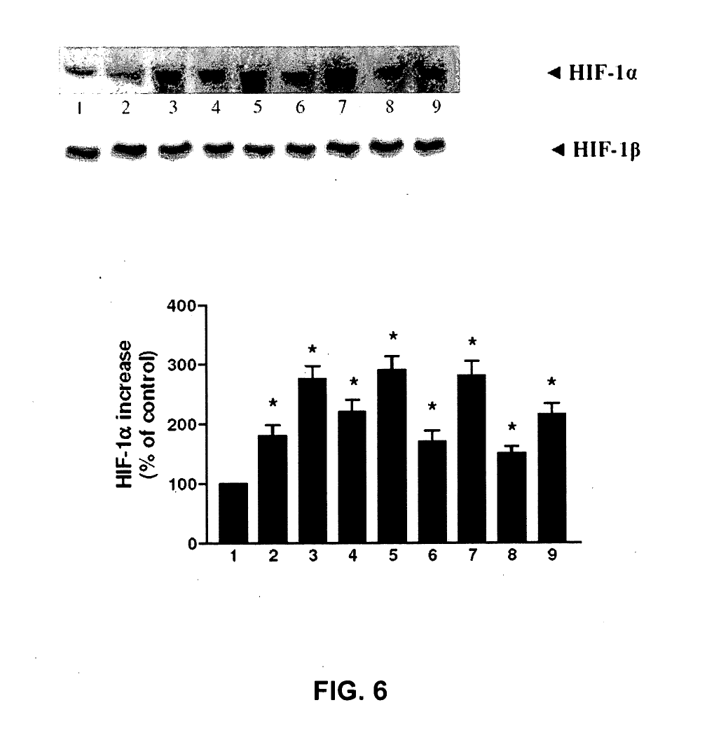

To evaluate the effect of ado on HIF-1α protein accumulation, PMA-treated U937, human macrophages and foam cells were incubated with adenosine (100 μM) for 4, 8 and 24 h. As PMA and oxLDL have been demonstrated to induce alone H IF-1α in normoxia, we performed the time course experiment both in normoxia and in hypoxia. In our experimental conditions, in PMA-treated U937 cells, under normoxia it was possible to detect only a slight band specific for HIF-1α protein poorly increased by adenosine after 24 hours (1.4 fold of increase evaluated through densitometric analysis, FIG. 4A). In contrast, a strong band specific for HIF-1α protein appear under hypoxic conditions starting from 4 h, that was significantly stimulated by adenosine and that was stable until 24 h (FIG. 4B). In human macrophages under normoxia the presence of HIF-1α was not observed, while the time of 4 h was optimal to evaluate adenosine stimulation in hypoxia (FIGS. 4C and 4D, respectively). Finally, in U937 derived foam cells the time course experiment in the presence of two different doses 50 and 100 μg/mL of oxLDL were performed. Again in normoxia, after treatment with 50 μg/mL of oxLDL, it was possible to detect only a slight band specific for HIF-1α protein at 24 h and this was slightly affected by adenosine (FIG. 4E). In contrast, a strong band specific for HIF-1α protein appear under hypoxic conditions which was increased by adenosine (100 μM) starting from 4 h, and was stable after 24 h and similar to that obtained by using 50 or 100 μg/mL of oxLDL (FIGS. 4F and 4G). Therefore, the concentration of 50 μg/mL at 4 h of hypoxia was chosen in order to study the effect of adenosine on HIF-1α protein accumulation in foam cells. To evaluate which adenosine receptor was involved in the adenosine induced HIF-1α protein accumulation foam cells were treated with selective antagonists of the adenosine receptors before addition of adenosine under hypoxic conditions. As shown in FIG. 5, the adenosine effect was partially antagonized by DPCPX, SCH 58261, MRE-2029F20 and MRE-3008F20 (100 nM) suggesting the involvement of A₁, A_{2A}, A_{2B} and A₃ adenosine receptors, respectively. Therefore, the effect of increasing concentrations of a series of high affinity agonists on HIF-1α accumulation was evaluated: cyclohexyl-adenosine (CHA; 10, 100 nM), 2-[p-(carboxyethyl)-phenethylamino]-NECA (CGS 21680; 500, 1000 nM), 1-deoxy-1-[6-{4-[(phenylcarbamoyl)-methoxy]phenylamino}-9H-purin-9-yl]-N-ethyl-β-D-ribofuranuronamide (10, 100 nM) and N⁶-(3iodobenzyl)-2-chloroadenosine-5′-N-methyluronamide (CI-IB-MECA; 10, 100 nM). As shown in FIG. 6, all the agonists were able to induce HIF-1α protein in foam cells. Analogous results were obtained in PMA-treated U937 cells and in human macrophages.

Knockdown of Adenosine Receptors by siRNA Treatment

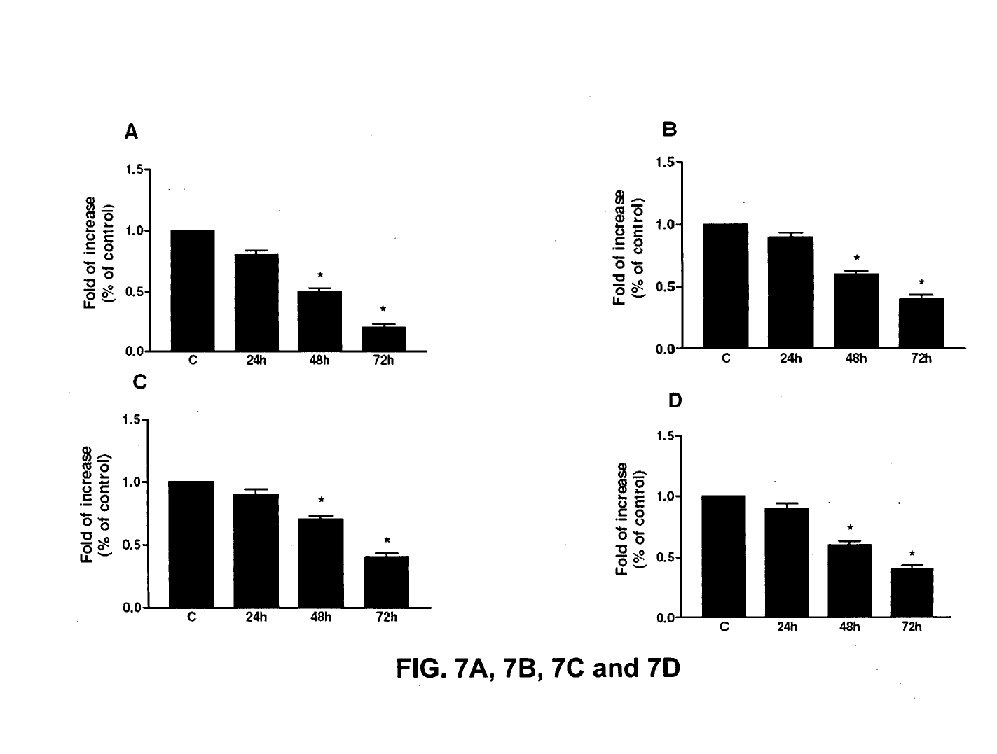

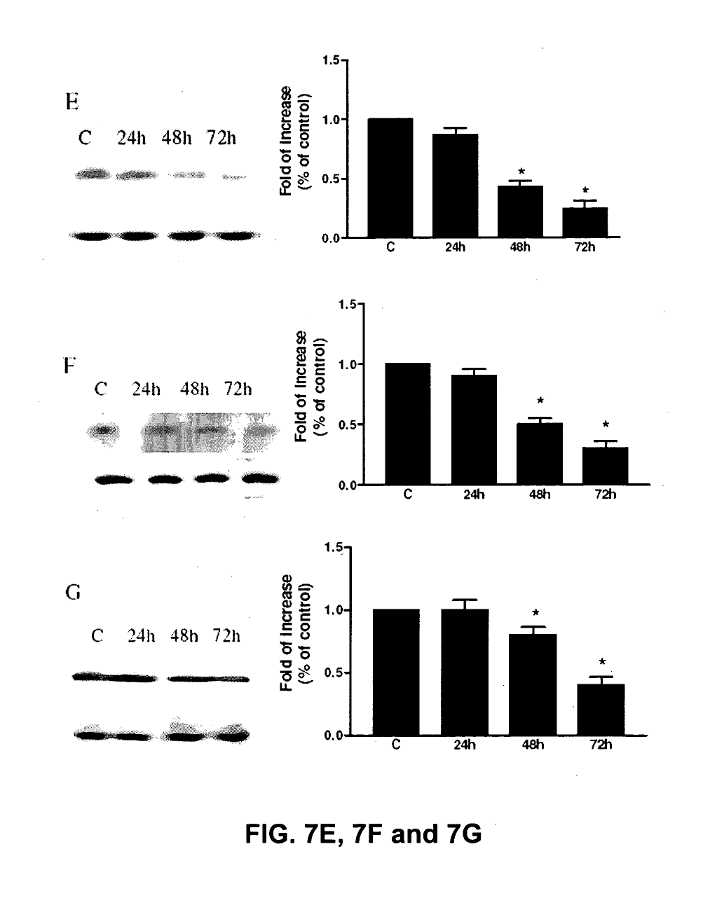

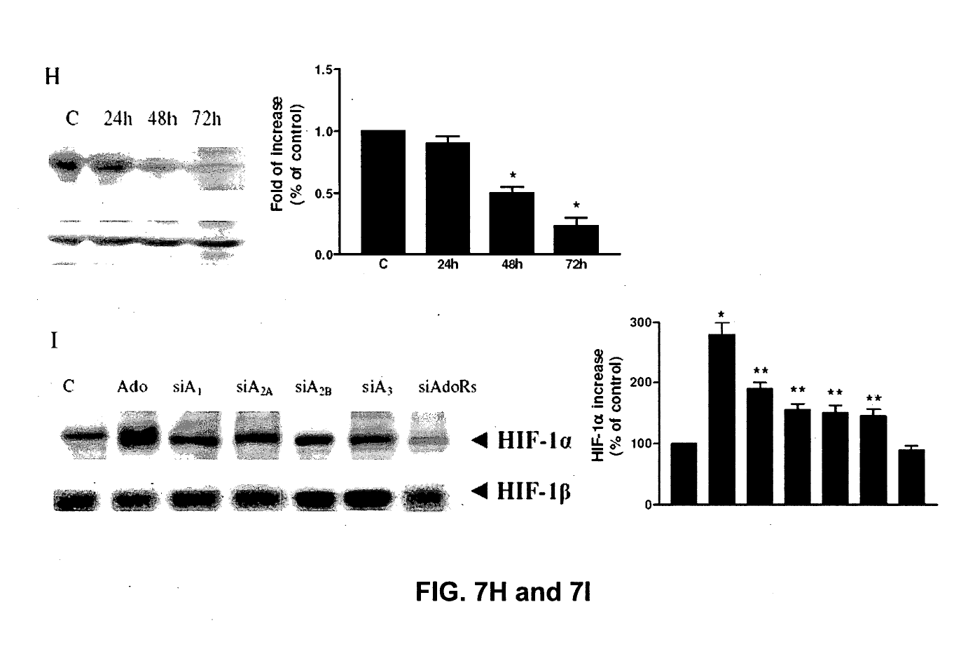

In order to further ascertain the involvement of the different receptor subtypes in the adenosine induced HIF1-α accumulation, each adenosine receptor was knocked-down using small interfering RNA (siRNA) leading to a transient silencing of A₁, A_{2A}, A_{2B} and A₃ receptors, respectively. Foam cells were transfected with siRNA targeting each adenosine subtype. After 48 and 72 h post transfection, adenosine receptor mRNAs (FIGS. 7A-7D, respectively) and protein levels were significantly reduced (FIGS. 7E-7H, respectively). Neither mock transfection nor transfection with a siRNA targeted to an irrelevant mRNA inhibited adenosine receptors expression. As shown in FIG. 71 treatment of the cells with the siRNA for A₁, A_{2A}, A_{2B} and A₃ subtypes for 72 h in hypoxic conditions reduced the effect of adenosine on HIF-1α modulation further supporting the role for all adenosine subtypes in this effect.

### Regulation of HIF-1α Protein Accumulation at Transcriptional Level

To study the molecular mechanism responsible for HIF-1α protein accumulation by adenosine, the nucleoside effect on HIF-1α mRNA expression was evaluated. Real-time RT-PCR experiments revealed that treatment of the cells with adenosine did not affect HIF-1α mRNA levels in normoxia while it induced a time-dependent increase of HIF-1α mRNA levels in hypoxia of 1.6±0.1, 1.9±0.1 and 1.5±0.1 fold after 4 h of treatment, respectively.

### Adenosine Receptors Induce VEGF Increase in Hypoxia

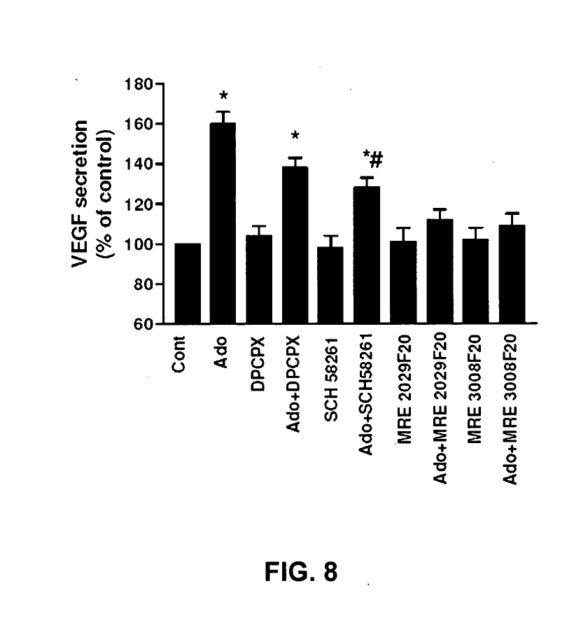

The effect of adenosine on VEGF production was tested in the supernatant of U937 derived foam cells at 24 h in hypoxic conditions. Adenosine (100 μM) increases VEGF levels by 165±10% and the effect was strongly reduced by MRE-2029F20 and MRE-3008F20 (100 nM) suggesting the involvement of A_{2B} and A₃ receptors, and was inhibited to lesser extent by the A_{2A} antagonist, SCH 58261 (FIG. 8). Moreover, treatment of the cells with siRNA of HIF-1α abrogated the increase in VEGF production induced by adenosine suggesting that the nucleoside was acting through HIF-1α modulation.

### A_{2B} Adenosine Receptor Induces IL-8 Increase in Hypoxia

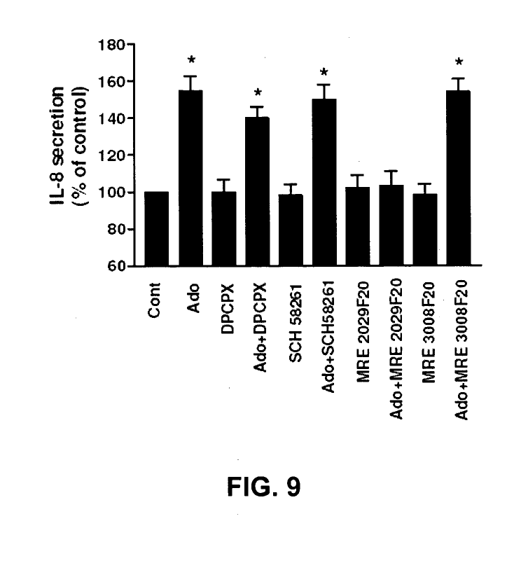

The effect of adenosine on IL-8 production was tested in the supernatant of U937 derived foam cells at 24 and 48 h in hypoxic conditions. Adenosine (100 μM) increases IL-8 levels by 158±10% and the effect was blocked by the A_{2B} antagonist MRE 2029F20 or A_{2B} silencing, but not by 100 nM DPCPX, SCH 58261 and MRE 3008F20, suggesting a selective effect for A_{2B} receptors (FIG. 9). A dose-response curve of the adenosine A_{2B} receptor agonist, 1-deoxy-1-[6-{4-[(phenylcarbamoyl)methoxy]phenylamino}-9H-purin-9-yl]-N-ethyl-β-D-ribofuranuronamide, reveal an EC₅₀ value of 58±6 nM for stimulation of IL-8 secretion suggesting the involvement of A_{2B} receptor subtype in this response. The effect of the adenosine A_{2B} receptor agonist (1 μM, 142±8% of IL-8 secretion) was completely blocked by the A_{2B} receptor antagonist MRE-2029F20. Finally, to investigate whether the IL-8 secretion induced by adenosine was mediated through the HIF-1α protein increase, the cells were treated with siRNA of HIF-1α before stimulation with adenosine. After 72 h of transfection, IL-8 secretion was not affected by HIF-1α silencing, suggesting that this effect induced by adenosine was not dependent by HIF-1α.

### Cholesterol/Cholesteryl Ester Quantitation in U937-Derived Foam Cells

Foam cells formation from U937 cells was evaluated by performing Cholesterol/Cholesteryl Ester quantitation. Exposure of PMA-treated U937 cells to oxidized LDL induced an increase of cholesterol from 0.137 to 0.200, cholesterol+cholesteryl ester (total cholesterol) from 0.205 to 0.443 and cholesteryl esters from 0.068 to 0.243.

### Oil Red O Staining in U937-Derived Foam Cells

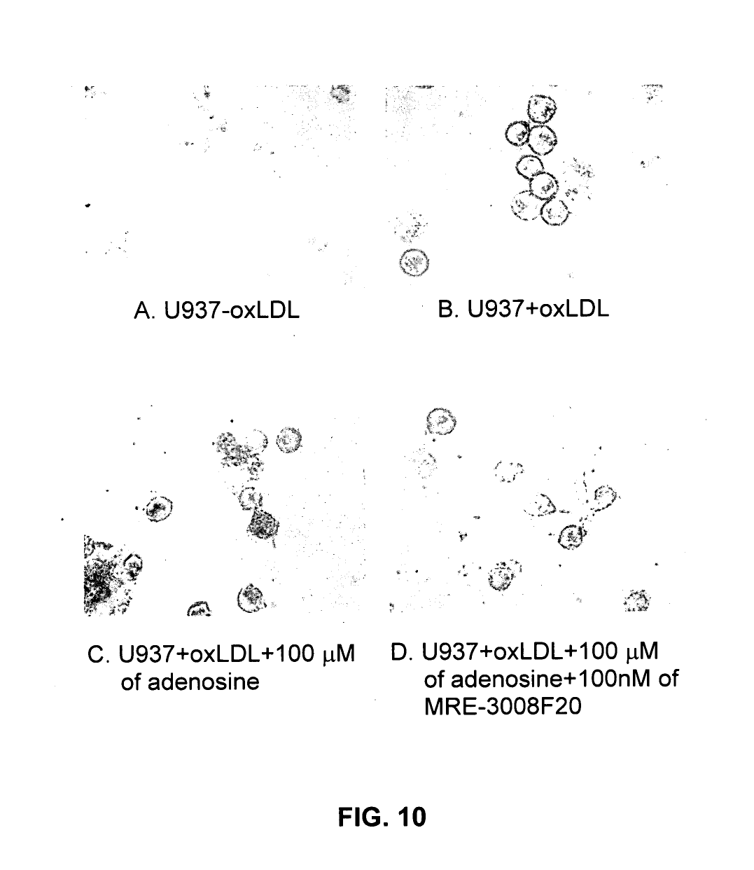

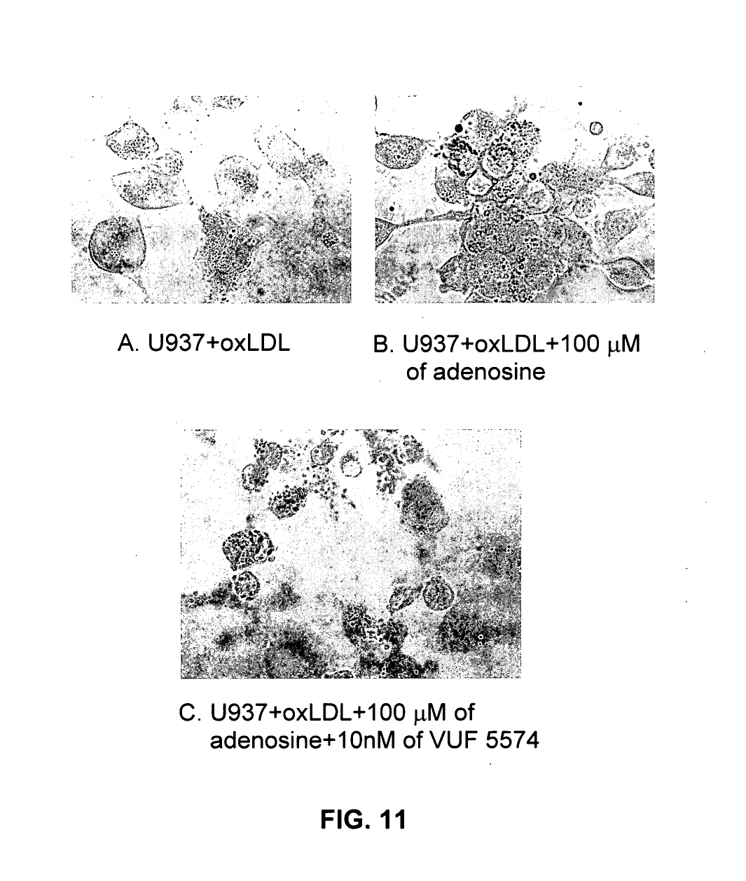

As shown in FIG. 10A, U937 cells without oxLDL do not contain high levels of neutral lipids and are not stained with Oil red O, a dye specific for neutral lipids. After treatment of PMA-treated U937 cells with 50 μg/mL of oxLDL for 24 h, an increase in foam cells characterized by large cytoplasmic lipid droplets was observed (FIG. 10B). This effect was increased after incubation with adenosine (100 μM, FIG. 10C). However, subsequent treatment with the adenosine A₃ receptor antagonists MRE-3008F20 (100 nM, FIG. 10D) and VUF 5574 (10 nM, FIG. 11C) blocked the foam cells formation.

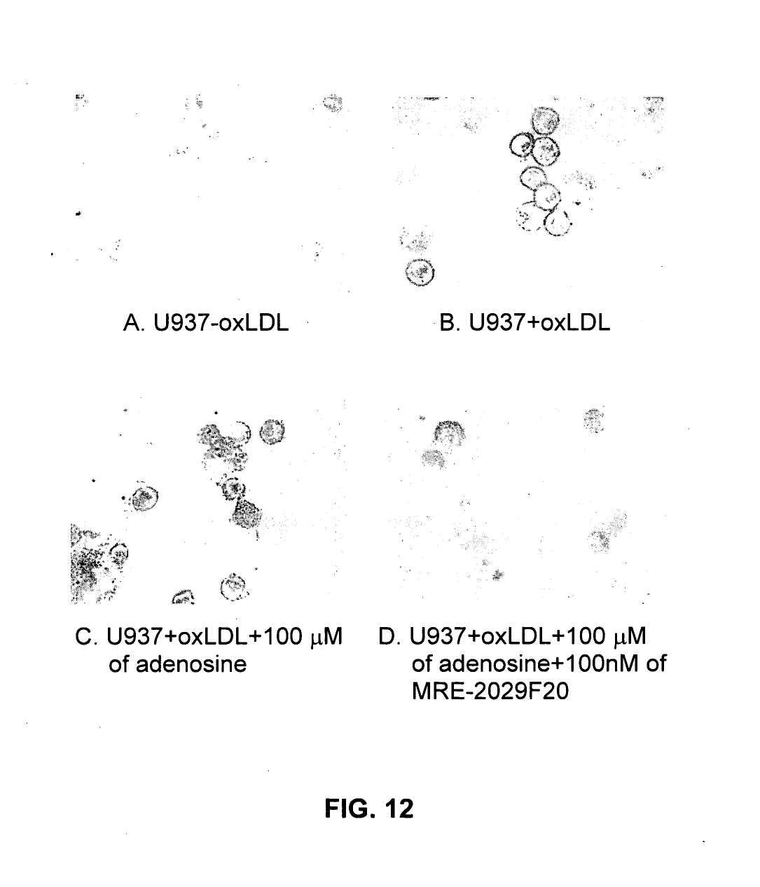

Likewise, as shown in FIG. 12D, treatment of U937 derived foam cells with the adenosine A_{2B} receptor antagonist, MRE-2029F20 (100 nM), also blocked the foam cells formation.

Altogether, these data demonstrate that activation of adenosine A_{2B} and A₃ receptors induces HIF-1α and VEGF accumulation in hypoxic conditions leading to foam cell formation and plaque angiogenesis and development, and that the A_{2B} receptor subtype is also responsible for IL-8 accumulation. Therefore, adenosine A_{2B} and A₃ receptor antagonists, or A_{2B}/A₃ dual antagonists, may be employed to block atherosclerotic plaque formation and progression.

1. (canceled)

2. A method for the prevention and treatment of atherosclerosis, which method comprises administering to a patient, in need thereof, a therapeutically effective amount of an adenosine A₃ receptor antagonist, or a pharmaceutically acceptable salt thereof.

3. A method according to claim 1 or 2, wherein an adenosine A₃ receptor antagonist is a compound of the formula 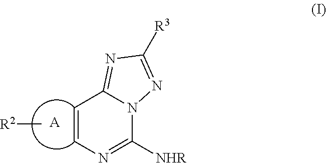
wherein
A is imidazole, pyrazole, or triazole;
R is —C(X)R¹, —C(X)—N(R¹)₂, —C(X)OR¹, —C(X)SR¹, —SO_{b}R¹, —SO_{b}OR¹, —SO_{b}SR¹, or —SO_{b}—N(R¹)₂;
R¹ is hydrogen, alkyl, substituted alkyl, cycloalkyl, substituted cycloalkyl, alkenyl, substituted alkenyl, alkynyl, substituted alkynyl, aryl, substituted aryl, heteroaryl, substituted heteroaryl, heterocyclyl, substituted heterocyclyl, wherein each R¹ can be the same or different; or, if linked to a nitrogen atom, then taken together with the nitrogen atom, —N(R¹)₂ forms an azetidine ring or a 5- or 6-membered heterocyclic ring optionally containing one or more additional heteroatoms selected from the group consisting of N, O, and S;
R² is hydrogen, alkyl, alkenyl, alkynyl, substituted alkyl, substituted alkenyl, substituted alkynyl, aralkyl, substituted aralkyl, aryl, substituted aryl, heteroaryl, or substituted heteroaryl;
R³ is furan, pyrrole, thiophene, benzofuran, benzypyrrole, benzothiophene, optionally substituted with 1 to 3 substituents selected from the group consisting of hydroxy, acyl, alkyl, alkoxy, alkenyl, alkynyl, substituted alkyl, substituted alkoxy, substituted alkenyl, substituted alkynyl, amino, aminoacyl, acyloxy, acylamino, aralkyl, aryl, substituted aryl, aryloxy, azido, carboxy, cyano, halo, nitro, heteroaryl, heteroaryloxy, heterocyclyl, heterocyclooxy, alkylthio, substituted alkylthio, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and trihalomethyl;
X is O, S, or NR'; and
b is 1 or 2;
or a pharmaceutically acceptable salt thereof.

4-10. (canceled)

11. A method according to claim 3, wherein
R represents —C(X)—N(R¹)₂ in which
R¹ is hydrogen, alkyl, substituted alkyl, alkenyl, substituted alkenyl, alkynyl, substituted alkynyl, aryl, substituted aryl, heteroaryl, substituted heteroaryl, heterocyclyl, substituted heterocyclyl, wherein each R¹ can be the same or different; or, if linked to a nitrogen atom, then taken together with the nitrogen atom, —N(R¹)₂ forms an azetidine ring or a 5- or 6-membered heterocyclic ring optionally containing one or more additional heteroatoms selected from the group consisting of N, O, and S;
X is O;
or a pharmaceutically acceptable salt thereof.

12. A method according to claim 11, wherein
R represents —C(O)—N(R¹)₂ in which each R¹ is different from each other, one being hydrogen;
A represents a pyrazole ring of the formula 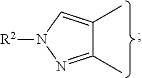
or a pharmaceutically acceptable salt thereof.

13. A method according to claim 12, wherein a compound of formula (I) has the following formula 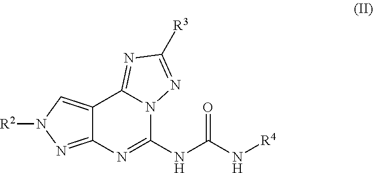
wherein
R² is hydrogen, alkyl, substituted alkyl, alkenyl, aralkyl, substituted aralkyl, heteroaryl, substituted heteroaryl or aryl;
R³ is furan;
R⁴ is aryl, substituted aryl, heteroaryl, substituted heteroaryl, heterocycle or substituted heterocycle;
or a pharmaceutically acceptable salt thereof.

14. A method according to claim 13, wherein the compound of formula (II) is selected from the group consisting of: 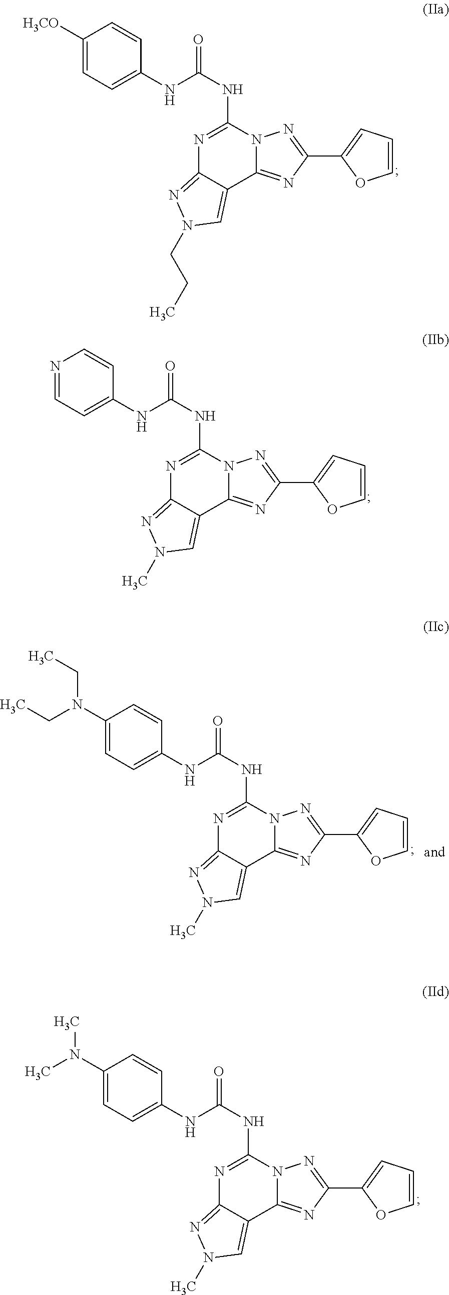
or in each case, a pharmaceutically acceptable salt thereof.

15. A method according to claim 3, wherein the compound of formula (I) is selected from the group consisting of:
5-{[(3-Chlorophenyl)amino]carbonyl}amino-8-methyl-2-(2-furyl)-pyrazolo[4,3-e]-1,2,4-triazolo[1,5-c]pyrimidine;
5-{[(4-Methoxyphenyl)amino]carbonyl}amino-8-methyl-2-(2-furyl)-pyrazolo[4,3-e]-1,2,4-triazolo[1,5-c]pyrimidine;
5-{[(3-Chlorophenyl)amino]carbonyl}amino-8-ethyl-2-(2-furyl)-pyrazolo[4,3-e]-1,2,4-triazolo[1,5-c]pyrimidine;
5-{[(4-Methoxyphenyl)amino]carbonyl}amino-8-ethyl-2-(2-furyl)-pyrazolo[4,3-e]-1,2,4-triazolo[1,5-c]pyrimidine;
5-{[(3-Chlorophenyl)amino]carbonyl}amino-8-propyl-2-(2-furyl)-pyrazolo[4,3-e]-1,2,4-triazolo[1,5-c]pyrimidine;
5-{[(4-Methoxyphenyl)amino]carbonyl}amino-8-propyl-2-(2-furyl)-pyrazolo[4,3-e]-1,2,4-triazolo[1,5-c]pyrimidine;
5-{[(3-Chlorophenyl)amino]carbonyl}amino-8-butyl-2-(2-furyl)-pyrazolo[4,3-e]-1,2,4-triazolo[1,5-c]pyrimidine;
5-{[(4-Methoxyphenyl)amino]carbonyl}amino-8-butyl-2-(2-furyl)-pyrazolo[4,3-e]-1,2,4-triazolo[1,5-c]pyrimidine;
5-{[(3-Chlorophenyl)amino]carbonyl}amino-8-isopentyl-2-(2-furyl)-pyrazolo[4,3-e]-1,2,4-triazolo[1,5-c]pyrimidine;
5-{[(4-Methoxyphenyl)amino]carbonyl}amino-8-isopentyl-2-(2-furyl)-pyrazolo[4,3-e]-1,2,4-triazolo[1,5-c]pyrimidine;
5-{[(3-Chlorophenyl)amino]carbonyl}amino-8-(2-isopentenyl)-2-(2-furyl)pyrazolo[4,3-e]-1,2,4-triazolo[1,5-c]pyrimidine;
5-{[(4-Methoxyphenyl)amino]carbonyl}amino-8-(2-isopentenyl)-2-(2-furyl)-pyrazolo[4,3-e]-1,2,4-triazolo[1,5-c]pyrimidine;
5-{[(3-Chlorophenyl)amino]carbonyl}amino-8-(2-phenylethyl)-2 (2-furyl)-pyrazolo[4,3-e]-1,2,4-triazolo[1,5-c]pyrimidine;
5-{[(4-Methoxyphenyl)amino]carbonyl}amino-8-(2-phenylethyl)-2-(2-furyl)-pyrazolo[4,3-e]-1,2,4-triazolo[1,5-c]pyrimidine;
5-{[(3-Chlorophenyl)amino]carbonyl}amino-8-(3-phenylpropyl)-2-(2-furyl)-pyrazolo[4,3-e]-1,2,4-triazolo[1,5-c]pyrimidine;
5-{[(4-Methoxyphenyl)amino]carbonyl}amino-8-(3-phenylpropyl)-2-(2-furyl)-pyrazolo[4,3-e]-1,2,4-triazolo[1,5-c]pyrimidine;
5-[(Benzyl)carbonyl]amino-8-isopentyl-2-(2-furyl)-pyrazolo[4,3-e]-1,2,4-triazolo[1,5-c]pyrimidine;
5-[(Benzyl)carbonyl]amino-8-(3-phenylpropyl)-2-(2-furyl)-pyrazolo[4,3-e]-1,2,4-triazolo[1,5-c]pyrimidine;
N-[4-(Diethylamino)phenyl]-N′-[2-(2-furyl)-8-methyl-8H-pyrazolo[4,3-e]-1,2,4-triazolo[1,5-c]pyrimidin-5-yl]urea;
N-[8-Methyl-2-(2-furyl)-8H-pyrazolo[4,3-e]-1,2,4-triazolo[1,5-c]pyrimidin-5-yl]-N′-[4-(dimethylamino)phenyl]urea;
N-[2-(2-Furyl)-8-methyl-8H-pyrazolo[4,3-e]-1,2,4-triazolo[1,5-c]pyrimidin-5-yl]-N′-[4-(morpholin-4-ylsulfonyl)phenyl]urea;
N-[2-(2-Furyl)-8-methyl-8H-pyrazolo[4,3-e]-1,2,4-triazolo[1,5-c]pyrimidin-5-yl]-N′-{4-[(4-methylpiperazin-1-yl)sulfonyl]phenyl}urea; and
N-[2-(2-Furyl)-8-methyl-8H-pyrazolo[4,3-e]-1,2,4-triazolo[1,5-c]pyrimidin-5-yl]-N′-pyridin-4-ylurea;
or a pharmaceutically acceptable salt thereof.

16. A method according to claim 1 or 2, wherein an adenosine A₃ receptor antagonist is a compound of the formula 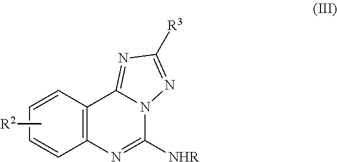
wherein
R is —C(X)R¹, —C(X)—N(R¹)₂, —C(X)OR¹, —C(X)SR¹, —SO_{b}R¹, —SO_{b}OR¹, —SO_{b}SR¹, or —SO_{b}—N(R¹)₂;
R¹ is hydrogen, alkyl, substituted alkyl, alkenyl, substituted alkenyl, alkynyl, substituted alkynyl, aryl, heteroaryl, substituted heteroaryl, or heterocyclyl, wherein each R¹ may be the same or different; or, if linked to a nitrogen atom, then taken together with the nitrogen atom, —N(R¹)₂ forms an azetidine ring or a 5- to 6-membered heterocyclic ring optionally containing one or more heteroatoms selected from N, O, and S;
R² is hydrogen, halogen, alkyl, alkenyl, alkynyl, substituted alkyl, substituted alkenyl, substituted alkynyl, aralkyl, substituted aralkyl, aryl, substituted aryl, heteroaryl or substituted heteroaryl;
R³ is furan, pyrrole, thiophene, benzofuran, benzypyrrole, benzothiophene, optionally substituted with 1 to 3 substituents selected from the group consisting of hydroxy, acyl, alkyl, alkoxy, alkenyl, alkynyl, substituted alkyl, substituted alkoxy, substituted alkenyl, substituted alkynyl, amino, aminoacyl, acyloxy, acylamino, alkaryl, aryl, substituted aryl, aryloxy, azido, carboxy, cyano, halo, nitro, heteroaryl, heteroaryloxy, heterocyclyl, heterocyclooxy, thioalkyl, substituted thioalkyl, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and trihalomethyl;
X is O, S, or NR¹;
b is 1 or 2;
or a pharmaceutically acceptable salt thereof.

17-23. (canceled)

24. A method according to claim 16, wherein
R represents —C(X)—N(R¹)₂ in which X is O; and wherein each R¹ can be the same or different;
or a pharmaceutically acceptable salt thereof.

25. A method according to claim 16, wherein the compound of formula (III) is selected from the group consisting of:
5-{[4-Methoxyphenyl)amino]carbonyl}amino-9-chloro-2-(2-furyl)-1,2,4-triazolo[1,5-c]quinazoline; and
5-{[3-Chlorophenyl)amino]carbonyl}amino-9-chloro-2-(2-furyl)-1,2,4-triazolo[1,5-c]quinazoline;
or a pharmaceutically acceptable salt thereof.

26. A method according to claim 2, wherein an adenosine A₃ receptor antagonist is a compound of the formula 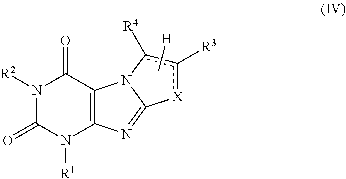
wherein
X is CH or N;
R¹ and R² are each independently hydrogen, alkyl, substituted alkyl, aralkyl, substituted aralkyl, alkenyl, substituted alkenyl, alkynyl, substituted alkynyl, aryl, or substituted aryl;
R³ is aryl, substituted aryl, alkyl, substituted alkyl, aralkyl, or substituted aralkyl;
R⁴ is hydrogen, alkyl, substituted alkyl, aralkyl, substituted aralkyl, aryl, or substituted aryl; and
one of the dashed lines represents a double bond and the other represents a single bond;
or a pharmaceutically acceptable salt thereof.

27. A method according to claim 26, wherein
R¹ is aralkyl;
R² is alkyl;
R⁴ is hydrogen, alkyl or substituted alkyl;
or a pharmaceutically acceptable salt thereof.

28. A method according to claim 26, wherein the adenosine A₃ receptor antagonist is a compound of the formula 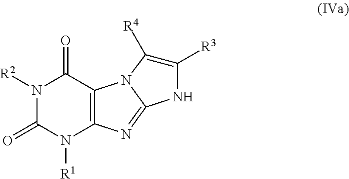
wherein
R¹ and R² are each independently hydrogen, alkyl, substituted alkyl, aralkyl, substituted aralkyl, alkenyl, substituted alkenyl, alkynyl, substituted alkynyl, aryl, or substituted aryl;
R³ is aryl, substituted aryl, alkyl, substituted alkyl, aralkyl, or substituted aralkyl;
R⁴ is hydrogen, alkyl, substituted alkyl, aralkyl, substituted aralkyl, aryl, or substituted aryl;
or a pharmaceutically acceptable salt thereof.

29. A method according to claim 28, wherein
R¹ is aralkyl;
R² is alkyl;
R⁴ is hydrogen, alkyl or substituted alkyl;
or a pharmaceutically acceptable salt thereof.

30. A method according to claim 26, wherein the adenosine A₃ receptor antagonist is a compound of the formula 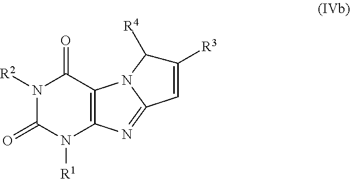
wherein
R¹ and R² are each independently hydrogen, alkyl, substituted alkyl, aralkyl, substituted aralkyl, alkenyl, substituted alkenyl, alkynyl, substituted alkynyl, aryl, or substituted aryl;
R³ is aryl, substituted aryl, alkyl, substituted alkyl, aralkyl, or substituted aralkyl;
R⁴ is hydrogen, alkyl, substituted alkyl, aralkyl, substituted aralkyl, aryl, or substituted aryl;
or a pharmaceutically acceptable salt thereof.

31. A method according to claim 30, wherein
R¹ is aralkyl;
R² is alkyl;
R⁴ is hydrogen, alkyl or substituted alkyl;
or a pharmaceutically acceptable salt thereof.

32. A method according to claim 26, wherein the adenosine A₃ receptor antagonist is selected from the group consisting of:
1-Benzyl-7-phenyl-3-propyl-1H-pyrrolo[1,2-f]purine-2,4(3H,6H)-dione;
1-Benzyl-7-phenyl-3-propyl-1H-imidazo[1,2-f]purine-2,4(3H,8H)-dione;
1-Benzyl-7-(4-methoxyphenyl)-3-propyl-1H-imidazo[1,2-f]purine-2,4(3H,8H)-dione;
1-Benzyl-7-(biphenyl-4-yl)-3-propyl-1H-imidazo[1,2-f]purine-2,4(3H,8H)-dione;
1-Benzyl-7-(4-fluorophenyl)-3-propyl-1H-imidazo[1,2-f]purine-2,4(3H,8H)-dione;
7-Phenyl-1,3-dipropyl-1H-imidazo[1,2-f]purine-2,4(3H,8H)-dione;
1,3-Diisobutyl-7-phenyl-1H-imidazo[1,2-f]purine-2,4(3H,8H)-dione;
1-Benzyl-7-methyl-3-propyl-1H-imidazo[1,2-f]purine-2,4(3H,8H)-dione;
1,3-Dimethyl-7-phenyl-1H-imidazo[1,2-f]purine-2,4(3H,8H)-dione;
7-(Biphenyl-4-yl)-1,3-dimethyl-1H-imidazo[1,2-f]purine-2,4(3H,8H)-dione;
7-(4-Chlorophenyl)-1,3-dimethyl-1H-imidazo[1,2-f]purine-2,4(3H,8H)-dione;
7-(4-Bromophenyl)-1,3-dimethyl-1H-imidazo[1,2-f]purine-2,4(3H,8H)-dione;
7-(4-Fluorophenyl)-1,3-dimethyl-1H-imidazo[1,2-f]purine-2,4(3H,8H)-dione;
7-(4-Methoxyphenyl)-1,3-dimethyl-1H-imidazo[1,2-f]purine-2,4(3H,8H)-dione;
1-Benzyl-7-methyl-3-propyl-1H-pyrrolo[1,2-f]purine-2,4(3H,6H)-dione;
1-Benzyl-7-ethyl-3-propyl-1H-pyrrolo[1,2-f]purine-2,4(3H,6H)-dione;
1-Benzyl-6,7-dimethyl-3-propyl-1H-pyrrolo[1,2-f]purine-2,4(3H,6H)-dione;
1-Benzyl-7-ethyl-3-propyl-1H-imidazo[1,2-f]purine-2,4(3H,8H)-dione;
1-Benzyl-7-isopropyl-3-propyl-1H-imidazo[1,2-f]purine-2,4(3H,8H)-dione;
1-Benzyl-7-t-butyl-3-propyl-1H-imidazo[1,2-f]purine-2,4(3H,8H)-dione;
1-Benzyl-7-cyclopropyl-3-propyl-1H-imidazo[1,2-f]purine-2,4(3H,8H)-dione;
1-Benzyl-7-cyclohexyl-3-propyl-1H-imidazo[1,2-f]purine-2,4(3H,8H)-dione;
1-Benzyl-6,7-dimethyl-3-propyl-1H-imidazo[1,2-f]purine-2,4(3H,8H)-dione;
1-Benzyl-7-ethyl-6-methyl-3-propyl-1H-imidazo[1,2-f]purine-2,4(3H,8H)-dione; and
1,3,7-Trimethyl-1H-imidazo[1,2-f]purine-2,4(3H,8H)-dione;
or a pharmaceutically acceptable salt thereof.

33. A method according to claim 2, wherein the method further comprises the prevention of stroke and heart attack.

34-35. (canceled)

36. A method for the prevention and treatment of atherosclerosis, which method comprises administering to a mammal, in need thereof, a therapeutically effective amount of a combination of an adenosine A₃ receptor antagonist, or a pharmaceutically acceptable salt thereof, and an adenosine A_{2B} receptor antagonist, or a pharmaceutically acceptable salt thereof.

37. A method according to claim 36, wherein the method further comprises the prevention of stroke and heart attack.
